(19) United States
(12) Patent Application Publication
(10) Pub. No.: US 2024/0321466 A1
(43) Pub. Date: Sep. 26, 2024

(54) HEALTH INFORMATION BASED COMMUNITIES AND KNOWLEDGE INCENTIVE SYSTEMS AND METHODS

(71) Applicants: KEVIN POTTIE, LONDON (CA); DANIEL LEGER, KOMOKA (CA)

(72) Inventors: KEVIN POTTIE, LONDON (CA); DANIEL LEGER, KOMOKA (CA)

(21) Appl. No.: 18/578,846

(22) PCT Filed: Jul. 14, 2022
(86) PCT No.: PCT/CA2022/051096

§ 371 Date: Jan. 12, 2024

Related U.S. Application Data
(60) Provisional application No. 63/266,070, filed on Dec. 28, 2021.
(60) Provisional application No. 63/222,077, filed on Jul. 15, 2021.

(51) Int. Cl. G16H 70/20; G06Q 30/0207; G16H 10/20; G16H 10/60; G16H 50/20; G16H 50/70

(52) U.S. Cl. CPC G16H 70/20; G06Q 30/0207; G16H 10/20; G16H 10/60; G16H 50/20; G16H 50/70

(57) ABSTRACT

Public misinterpretation of Internet health information represents a major liability in terms of both public inferences, health professional information processing, and costly. A system acquiring recommendations from “peer” level issuers of health and medical recommendations that provides portals allowing medical professionals to maintain their knowledge and the general public to acquire recommendations in plain language would help address these issues. Such a system would allow a medical professional to direct patients to this online source and track their usage it, allow medical certification authorities to track medical professional knowledge acquisition and establish time expiring ratings of current knowledge. The system ingests health recommendations and ensures they reach the public in the form of personalized plain language messages whilst allowing the local nature of inferences, influences, and knowledge, to factor into individual decision making through geographical clusters of knowledge.

### CROSS-REFERENCE TO RELATED APPLICATIONS

This patent application claims the benefit of priority as a 371 National Phase Entry application of PCT/CA2022/051096 filed Jul. 14, 2022; which itself claims the benefit of priority from U.S. Provisional Patent Application No. 63/222,077 filed Jul. 15, 2021; and the benefit of priority from U.S. Provisional Patent Application No. 63/266,070 filed Dec. 28, 2021; the entire contents of each being incorporated herein by reference.

### FIELD OF THE INVENTION

This patent application relates to health information and more particularly to processing health information, public and provider inferences, and recommendations generated by multiple sources to provide medical professionals, medical personnel, patients, caregivers, health consumers and the general public with improved information and systems/methods for tracking the user's activities.

### BACKGROUND OF THE INVENTION

Published health information and published health recommendations) provides an inferential framework within which users, from medical professionals to the general public, make choices and decisions with respect to a range of matters ranging from aspects of their diet, lifestyle to prevention and treatment of medical conditions, pharmaceutical choices etc. Inferences are social products whose truth is understood through action.

However, whilst the Internet has made a wealth of information accessible to users it has also led to propagation of poor information, difficult to understand information and information overload, etc. For instance, users in the general public may be unaware that the website offering health advise is sponsored by a pharmaceutical company and seeks to push their products or that the advice is being given by someone with no medical training or background or without application of scientific or evidence-based medicine appraisals. For medical professionals, maintaining their knowledge of evidence-based recommendations in an up-to-date manner or counter-advising patients who have absorbed poor advice/knowledge can be time consuming.

Published health information may or may not exist in reliable and consistent plain language translations. Misinterpretation of internet health information represents a major liability in terms of both public inferences and even health professional information processing. Public misinterpretation is costly; for example, fear of unfounded side effects with COVID-19 vaccines reduced vaccine uptake has left many populations unvaccinated and vulnerable to COVID-19 virus infection.

Indeed, events such as the COVID-19 pandemic have further highlighted these and other issues where actionable statements and recommendations emerge at a rapid pace, may change frequently, conflicting advice is provided online, physical physician visits severely limited, etc. Accordingly, it would be beneficial to provide a system which acquires recommendations from one or more “peer” level issuers of health and medical recommendations and provides portals allowing medical professionals to maintain their knowledge up to date in an efficient manner and the general public users to acquire recommendations in plain language. It would be further beneficial for the system to provide a medical professional with the ability to direct patients to this online source and their subsequent access to it, for medical certification authorities to track medical professional knowledge acquisition and establish time expiring ratings of current knowledge, etc.

It would be further beneficial for the system to exploit artificial intelligence to establish the content and ensure it reaches the public in a form of personalized messages so that different users receive personalized content based upon their user profile(s).

It would be further beneficial, given the local nature of inferences, influences, and knowledge, for the system to exploit location-based information allowing these aspects of individual decision making to be enhanced through the development of local or geographical clusters of knowledge for the public and practitioners.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to health information and more particularly to processing health information, public and provider inferences, and recommendations generated by multiple sources to provide medical professionals, medical personnel, patients, caregivers, health consumers and the general public with improved plain language information and systems/methods for tracking the user's activities.

In accordance with an embodiment of the invention there is provided a system comprising:
a memory accessible to one or more microprocessors storing a database comprising a plurality of recommendations;
another memory accessible to the one or more microprocessors storing another database comprising user data relating to a set of users;
a further memory accessible to the one or more microprocessors storing computer executable instructions which when executed by the one or more microprocessors configure the one or more microprocessors to execute a process comprising:
   ingest a recommendation;
   process the ingested recommendation with at least a plurality of artificial intelligence (AI) processes and a plurality of machine learning (ML) processes;
   generate a narrative from the processed ingested recommendation;
   generate an expert commentary targeted to medical professionals;
   generate at least one of a plain language recommendation and a plain language actionable message statement targeted to the medical professionals;
   generate at least one of another plain language recommendation and another plain language actionable message statement targeted to non-medical professional users;
   generate at least one of a local relevance question and a local truth question to present to each user accessing the ingested recommendation;
   generate metadata for storage within one or more web servers for use in generating and rendering to a user the ingested recommendation upon an electronic device associated with the user.

In accordance with an embodiment of the invention there is provided a method comprising: ingesting a recommendation;
processing the ingested recommendation with at least a plurality of artificial intelligence (AI) processes and a plurality of machine learning (ML) processes;
generating a narrative from the processed ingested recommendation;
generating an expert commentary targeted to medical professionals;
generating at least one of a plain language recommendation and a plain language actionable message statement targeted to the medical professionals;
generating at least one of another plain language recommendation and another plain language actionable message statement targeted to non-medical professional users;
generating at least one of a local relevance question and a local truth question to present to each user accessing the ingested recommendation;
generating metadata for storage within one or more web servers for use in generating and rendering to a user the ingested recommendation upon an electronic device associated with the user.

In accordance with an embodiment of the invention there is provided a method comprising:
providing a user with credits, each credit associated with the user accessing and engaging within a software application a recommendation; wherein
the credits are fed back from the software application to a professional association associated with the user; and
the credits are employed either in establishing an accreditation of the user with the professional association or in completing a requirement for advancement of the user with the professional association.

In accordance with an embodiment of the invention there is provided a method comprising:
providing a user with credits, each credit associated with the user accessing and engaging within a software application a recommendation; wherein
the credits are fed back from the software application to an electronic medical record of the user; and
the credits are subsequently employed by a medical professional to establish a history that defines the user's background knowledge or experience with “plain language” recommendations.

In accordance with an embodiment of the invention there is provided a method comprising:
providing a user with credits, each credit associated with the user accessing and engaging within a software application a recommendation; wherein
the credits are linked from the software application to another software application; and
the user can establish at least one of:
   at least one of a financial reward, a financial discount, and a financial credit to employ in respect of purchasing at least one of a service and a product; and
   an option with respect of at least one of a service and a product which is only available to users exceeding a specific credit threshold.

In accordance with an embodiment of the invention there is provided a system comprising:
a memory accessible to one or more microprocessors storing a database comprising a plurality of recommendations;
another memory accessible to the one or more microprocessors storing another database comprising user data relating to a set of users;
a further memory accessible to the one or more microprocessors storing computer executable instructions which when executed by the one or more microprocessors configure the one or more microprocessors to execute a process comprising:
   managing documentation for each cluster of a plurality of clusters;
   managing communication between users for each cluster of the plurality of users; and
   establish one or more knowledge-based communities for each cluster of the plurality of clusters; wherein
each cluster of the plurality of clusters is established in dependence upon at least one of:
   geographic locations of each user of a plurality of users accessing the system; and
   profiles of each user of the plurality of users accessing the systems.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts an exemplary network environment within which configurable electrical devices according to and supporting embodiments of the invention may be deployed and operate; and

FIG. 2 depicts an exemplary wireless portable electronic devices supporting communications to a network such as depicted in FIG. 1 and embodiments of the invention;

FIGS. 3A and 3B depict exemplary process flows for onboarding recommendations within a system according to an embodiment of the invention;

FIGS. 4-6 depict exemplary recommendation ingestion and user directed outputs or inferences from a system according to an embodiment of the invention where a public and provider inference feedback loop is established by a system according to an embodiment of the invention to develop user truth(s) for the health recommendation(s).

### DETAILED DESCRIPTION

The present invention is directed to health information and more particularly to processing health information, public and provider inferences, and recommendations generated by multiple sources to provide medical professionals, medical personnel, patients, caregivers, health consumers and the general public with improved information and systems/methods for tracking the user's activities.

The ensuing description provides representative embodiment(s) only, and is not intended to limit the scope, applicability, or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment,” “an embodiment,” “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purposes only. It is to be understood that where the claims or specification refer to “a” or “an” element, such reference is not to be construed as there being only one of that element. It is to be understood that where the specification states that a component feature, structure, or characteristic “may,” “might,” “can” or “could” be included, that particular component, feature, structure, or characteristic is not required to be included.

Reference to terms such as “left,” “right,” “top,” “bottom,” “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users.

Reference to terms “including,” “comprising,” “consisting,” and grammatical variants thereof do not preclude the addition of one or more components, features, steps, integers, or groups thereof and that the terms are not to be construed as specifying components, features, steps, or integers. Likewise, the phrase “consisting essentially of,” and grammatical variants thereof, when used herein is not to be construed as excluding additional components, steps, features integers or groups thereof but rather that the additional features, integers, steps, components, or groups thereof do not materially alter the basic and novel characteristics of the claimed composition, device or method. If the specification or claims refer to “an additional” element, that does not preclude there being more than one of the additional element.

A “wireless standard” as used herein and throughout this disclosure, refer to, but is not limited to, a standard for transmitting signals and/or data through electromagnetic radiation which may be optical, radio-frequency (RF) or microwave although typically RF wireless systems and techniques dominate. A wireless standard may be defined globally, nationally, or specific to an equipment manufacturer or set of equipment manufacturers. Dominant wireless standards at present include, but are not limited to IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, Bluetooth, Wi-Fi, Ultra-Wideband and WiMAX. Some standards may be a conglomeration of sub-standards such as IEEE 802.11 which may refer to, but is not limited to, IEEE 802.1a, IEEE 802.11b, IEEE 802.11g, or IEEE 802.11n as well as others under the IEEE 802.11 umbrella.

A “wired standard” as used herein and throughout this disclosure, generally refer to, but is not limited to, a standard for transmitting signals and/or data through an electrical cable discretely or in combination with another signal. Such wired standards may include, but are not limited to, digital subscriber loop (DSL), Dial-Up (exploiting the public switched telephone network (PSTN) to establish a connection to an Internet service provider (ISP)), Data Over Cable Service Interface Specification (DOCSIS), Ethernet, Gigabit home networking (G.hn), Integrated Services Digital Network (ISDN), Multimedia over Coax Alliance (MoCA), and Power Line Communication (PLC, wherein data is overlaid to AC/DC power supply). In some embodiments a “wired standard” may refer to, but is not limited to, exploiting an optical cable and optical interfaces such as within Passive Optical Networks (PONs) for example.

A “user” as used herein may refer to, but is not limited to, an individual or group of individuals. This includes, private individuals, employees of organizations and/or enterprises, members of community organizations, members of charity organizations, men, women, and children. In its broadest sense the user may further include, but not be limited to, mechanical systems, robotic systems, android systems, etc. that may be characterised by an ability to exploit or implement one or more embodiments of the invention. A user may also be associated through one or more accounts and/or profiles with one or more of a service provider, third party provider, enterprise, social network, social media etc. via a dashboard, web service, website, software plug-in, software application, and graphical user interface. A user may include, but not be limited to, a medical professional, medical personnel, a patient, a caregiver, a health consumer and a member of the general public A user may be a member of the general public, who may also be referred to as a subscriber within the following specification on the basis of subscribing to a service according to an embodiment of the invention or a medical professional, who may also be referred to as a provider subscriber within the following specification on the basis of subscribing to a service according to an embodiment of the invention.

A “sensor” as used herein may refer to, but is not limited to, a transducer providing an electrical output generated in dependence upon a magnitude of a measure and selected from the group comprising, but is not limited to, biometric sensors, environmental sensors, medical sensors, biological sensors, chemical sensors, ambient environment sensors, position sensors, motion sensors, thermal sensors, infrared sensors, visible sensors, RFID sensors, and medical testing and diagnosis devices.

A “portable electronic device” (PED) as used herein and throughout this disclosure, refers to a wireless device used for communications and other applications that requires a battery or other independent form of energy for power. This includes devices, but is not limited to, such as a cellular telephone, smartphone, personal digital assistant (PDA), portable computer, pager, portable multimedia player, portable gaming console, laptop computer, tablet computer, a wearable device, and an electronic reader.

A “fixed electronic device” (FED) as used herein and throughout this disclosure, refers to a wireless and/or wired device used for communications and other applications that requires connection to a fixed interface to obtain power. This includes, but is not limited to, a laptop computer, a personal computer, a computer server, a kiosk, a gaming console, a digital set-top box, an analog set-top box, an Internet enabled appliance, an Internet enabled television, and a multimedia player.

A “server” as used herein, and throughout this disclosure, refers to one or more physical computers co-located and/or geographically distributed running one or more services as a host to users of other computers, PEDs, FEDs, etc. to serve the client needs of these other users. This includes, but is not limited to, a database server, file server, mail server, print server, web server, gaming server, or virtual environment server.

An “application” (commonly referred to as an “app”) as used herein may refer to, but is not limited to, a “software application”, an element of a “software suite”, a computer program designed to allow an individual to perform an activity, a computer program designed to allow an electronic device to perform an activity, and a computer program designed to communicate with local and/or remote electronic devices. An application thus differs from an operating system (which runs a computer), a utility (which performs maintenance or general-purpose chores), and a programming tools (with which computer programs are created). Generally, within the following description with respect to embodiments of the invention an application is generally presented in respect of software permanently and/or temporarily installed upon a PED and/or FED.

An “enterprise” as used herein may refer to, but is not limited to, a provider of a service and/or a product to a user, customer, or consumer. This includes, but is not limited to, a retail outlet, a store, a market, an online marketplace, a manufacturer, an online retailer, a charity, a utility, and a service provider. Such enterprises may be directly owned and controlled by a company or may be owned and operated by a franchisee under the direction and management of a franchiser.

A “service provider” as used herein may refer to, but is not limited to, a third-party provider of a service and/or a product to an enterprise and/or individual and/or group of individuals and/or a device comprising a microprocessor. This includes, but is not limited to, a retail outlet, a store, a market, an online marketplace, a manufacturer, an online retailer, a utility, an own brand provider, and a service provider wherein the service and/or product is at least one of marketed, sold, offered, and distributed by the enterprise solely or in addition to the service provider.

A “third party” or “third party provider” as used herein may refer to, but is not limited to, a so-called “arm's length” provider of a service and/or a product to an enterprise and/or individual and/or group of individuals and/or a device comprising a microprocessor wherein the consumer and/or customer engages the third party but the actual service and/or product that they are interested in and/or purchase and/or receive is provided through an enterprise and/or service provider.

“Biometric” information as used herein may refer to, but is not limited to, data relating to a user characterised by data relating to a subset of conditions including, but not limited to, their environment, medical condition, biological condition, physiological condition, chemical condition, ambient environment condition, position condition, neurological condition, drug condition, and one or more specific aspects of one or more of these said conditions. Accordingly, such biometric information may include, but not be limited, blood oxygenation, blood pressure, blood flow rate, heart rate, temperate, fluidic pH, viscosity, particulate content, solids content, altitude, vibration, motion, perspiration, EEG, ECG, energy level, etc. In addition, biometric information may include data relating to physiological characteristics related to the shape and/or condition of the body wherein examples may include, but are not limited to, fingerprint, facial geometry, baldness, DNA, hand geometry, odour, and scent. Biometric information may also include data relating to behavioral characteristics, including but not limited to, typing rhythm, gait, and voice.

“User information” as used herein may refer to, but is not limited to, user behavior information and/or user profile information. It may also include a user's biometric information, an estimation of the user's biometric information, or a projection/prediction of a user's biometric information derived from current and/or historical biometric information.

A “wearable device” or “wearable sensor” relates to miniature electronic devices that are worn by the user including those under, within, with or on top of clothing and are part of a broader general class of wearable technology which includes “wearable computers” which in contrast are directed to general or special purpose information technologies and media development. Such wearable devices and/or wearable sensors may include, but not be limited to, smartphones, smart watches, e-textiles, smart shirts, activity trackers, smart glasses, environmental sensors, medical sensors, biological sensors, physiological sensors, chemical sensors, ambient environment sensors, position sensors, neurological sensors, drug delivery systems, medical testing and diagnosis devices, and motion sensors.

“Electronic content” (also referred to as “content” or “digital content”) as used herein may refer to, but is not limited to, any type of content that exists in the form of digital data as stored, transmitted, received and/or converted wherein one or more of these steps may be analog although generally these steps will be digital. Forms of digital content include, but are not limited to, information that is digitally broadcast, streamed, or contained in discrete files. Viewed narrowly, types of digital content include popular media types such as MP3, JPG, AVI, TIFF, AAC, TXT, RTF, HTML, XHTML, PDF, XLS, SVG, WMA, MP4, FLV, and PPT, for example, as well as others, see for example http://en.wikipedia.org/wiki/List_of_file_formats. Within a broader approach digital content mat include any type of digital information, e.g., digitally updated weather forecast, a global positioning system (GPS) map, an eBook, a photograph, a video, a Vine™, a blog posting, a Facebook™ posting, a Twitter™ tweet, online TV, etc. The digital content may be any digital data that is at least one of generated, selected, created, modified, and transmitted in response to a user request, said request may be a query, a search, a trigger, an alarm, and a message for example.

A “profile” as used herein, and throughout this disclosure, refers to a computer and/or microprocessor readable data file comprising data relating to settings and/or limits of an adult device. Such profiles may be established by a manufacturer/supplier/provider of a device, service, etc. or they may be established by a user through a user interface for a device, a service, or a PED/FED in communication with a device, another device, a server, or a service provider etc.

A “computer file” (commonly known as a file) as used herein, and throughout this disclosure, refers to a computer resource for recording data discretely in a computer storage device, this data being electronic content. A file may be defined by one of different types of computer files, designed for different purposes. A file may be designed to store electronic content such as a written message, a video, a computer program, or a wide variety of other kinds of data. Some types of files can store several types of information at once. A file can be opened, read, modified, copied, and closed with one or more software applications an arbitrary number of times. Typically, files are organized in a file system which can be used on numerous different types of storage device exploiting different kinds of media which keeps track of where the files are located on the storage device(s) and enables user access. The format of a file is defined by its content since a file is solely a container for data, although, on some platforms the format is usually indicated by its filename extension, specifying the rules for how the bytes must be organized and interpreted meaningfully. For example, the bytes of a plain text file are associated with either ASCII or UTF-8 characters, while the bytes of image, video, and audio files are interpreted otherwise. Some file types also allocate a few bytes for metadata, which allows a file to carry some basic information about itself.

“Metadata” as used herein, and throughout this disclosure, refers to information stored as data that provides information about other data. Many distinct types of metadata exist, including but not limited to, descriptive metadata, structural metadata, administrative metadata, reference metadata, inferential metadata, and statistical metadata. Descriptive metadata may describe a resource for purposes such as discovery and identification and may include, but not be limited to, elements such as title, abstract, author, and keywords. Structural metadata relates to containers of data and indicates how compound objects are assembled and may include, but not be limited to, how pages are ordered to form chapters, and typically describes the types, versions, relationships, and other characteristics of digital materials. Administrative metadata may provide information employed in managing a resource and may include, but not be limited to, when and how it was created, file type, technical information, and who can access it. Reference metadata may describe the contents and quality of statistical data whereas statistical metadata may also describe processes that collect, process, or produce statistical data. Inferential metadata as used herein refers to, but is not limited to, data relating to public and/or provider inferences and knowledge related to a local relevance and/or a local truth for one or more health recommendations. Statistical metadata may also be referred to as process data.

“Plain language translation” as used herein, and throughout this disclosure, refers to easy-to-read and easy-to-understand translations of health information and recommendations. The target of these method and processes are both health professionals and the public and “plain language” is a core content and process ingredient to build consistent and convergent interpretation of rated health information. Benjamin in his seminal work, “The role of the translator,” provides an in-depth description of the process involved in the translation and mass production of works. In this same vein, we consider the plain language translation process within our method and processes as core in creating health professional and public interpretation. As Marshall Mcluhan writes “the medium is the message” or in our case the method, we consider our use plain language as integral to the process.

An “artificial intelligence system” (referred to hereafter as artificial intelligence, AI) as used herein, and throughout disclosure, refers to machine intelligence or machine learning in contrast to natural intelligence. An AI may refer to analytical, human inspired, or humanized artificial intelligence. An AI may refer to the use of one or more machine learning algorithms and/or processes. An AI may employ one or more of an artificial network, decision trees, support vector machines, Bayesian networks, and genetic algorithms. An AI may employ a training model or federated learning.

“Machine learning” (ML) or more specifically machine learning processes as used herein refers to, but is not limited, to programs, algorithms, or software tools, which allow a given device or program to learn to adapt its functionality based on information processed by it or by other independent processes. These learning processes are in practice, gathered from the result of said process which produce data and or algorithms that lend themselves to prediction. This prediction process allows ML-capable devices to behave according to guidelines initially established within its own programming but evolved as a result of the ML. A machine learning algorithm or machining learning process as employed by an AI may include, but not be limited to, supervised learning, unsupervised learning, cluster analysis, reinforcement learning, feature learning, sparse dictionary learning, anomaly detection, association rule learning, inductive logic programming.

### Health Information Based Communities and Knowledge Incentive Systems

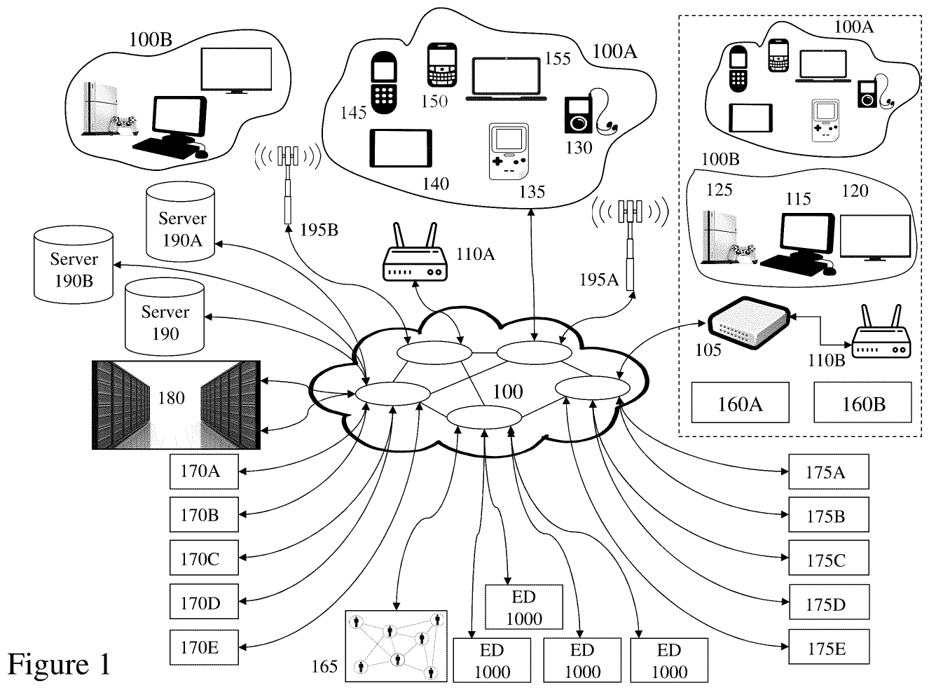

Referring to FIG. 1 there is depicted a Network 100 within which embodiments of the invention may be employed supporting Recommendation Systems, Applications and Platforms (R-SAPs) according to embodiments of the invention. Such R-SAPs, for example, supporting multiple communication channels, dynamic filtering, etc. As shown first and second user groups 100A and 100B respectively interface to a telecommunications Network 100. Within the representative telecommunication architecture, a remote central exchange 180 communicates with the remainder of a telecommunication service providers network via the Network 100 which may include for example long-haul OC-48/OC-192 backbone elements, an OC-48 wide area network (WAN), a Passive Optical Network, and a Wireless Link. The central exchange 180 is connected via the Network 100 to local, regional, and international exchanges (not shown for clarity) and therein through Network 100 to first and second cellular APs 195A and 195B respectively which provide Wi-Fi cells for first and second user groups 100A and 100B, respectively. Also connected to the Network 100 are first and second Wi-Fi nodes 110A and 110B, the latter of which being coupled to Network 100 via router 105. Second Wi-Fi node 110B is associated with R-SAP 160, e.g., Caretek™, comprising other first and second user groups 100A and 100B. Second user group 100B may also be connected to the Network 100 via wired interfaces including, but not limited to, DSL, Dial-Up, DOCSIS, Ethernet, G.hn, ISDN, MoCA, PON, and Power line communication (PLC) which may or may not be routed through a router such as router 105.

Within the cell associated with first AP 110A the first group of users 100A may employ a variety of PEDs including for example, laptop computer 155, portable gaming console 135, tablet computer 140, smartphone 150, cellular telephone 145 as well as portable multimedia player 130. Within the cell associated with second AP 110B are the second group of users 100B which may employ a variety of FEDs including for example gaming console 125, personal computer 115 and wireless/Internet enabled television 120 as well as cable modem 105. First and second cellular APs 195A and 195B respectively provide, for example, cellular GSM (Global System for Mobile Communications) telephony services as well as 3G and 4G evolved services with enhanced data transport support. Second cellular AP 195B provides coverage in the exemplary embodiment to first and second user groups 100A and 100B. Alternatively the first and second user groups 100A and 100B may be geographically disparate and access the Network 100 through multiple APs, not shown for clarity, distributed geographically by the network operator or operators. First cellular AP 195A as show provides coverage to first user group 100A and environment 170, which comprises second user group 100B as well as first user group 100A. Accordingly, the first and second user groups 100A and 100B may according to their particular communications interfaces communicate to the Network 100 through one or more wireless communications standards such as, for example, IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, and IMT-1000. It would be evident to one skilled in the art that many portable and fixed electronic devices may support multiple wireless protocols simultaneously, such that for example a user may employ GSM services such as telephony and SMS and Wi-Fi/WiMAX data transmission, VOIP and Internet access. Accordingly, portable electronic devices within first user group 100A may form associations either through standards such as IEEE 802.15 or Bluetooth as well in an ad-hoc manner.

Also connected to the Network 100 are Social Networks (SOCNETS) 165, first and second service providers 170A and 170B respectively, first and second third party service providers 170C and 170D respectively, and a user 170E. Also connected to the Network 100 are first and second enterprises 175A and 175B respectively, first and second organizations 175C and 175D respectively, and a government entity 175E. Also depicted are first and second servers 190A and 190B may host according to embodiments of the inventions multiple services associated with a provider of contact management systems and contact management applications/platforms (R-SAPs); a provider of a SOCNET or Social Media (SOME) exploiting R-SAP features; a provider of a SOCNET and/or SOME not exploiting R-SAP features; a provider of services to PEDS and/or FEDS; a provider of one or more aspects of wired and/or wireless communications; an R-SAP 160, e.g. Caretek™, exploiting R-SAP features; license databases; content databases; image databases; content libraries; customer databases; websites; and software applications for download to or access by FEDs and/or PEDs exploiting and/or hosting R-SAP features. First and second primary content servers 190A and 190B may also host for example other Internet services such as a search engine, financial services, third party applications and other Internet based services.

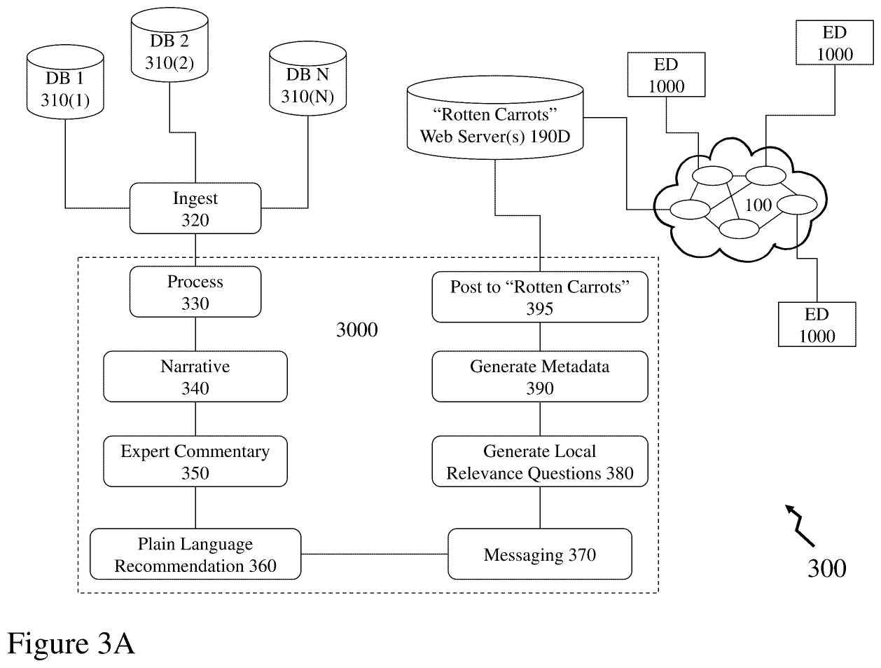

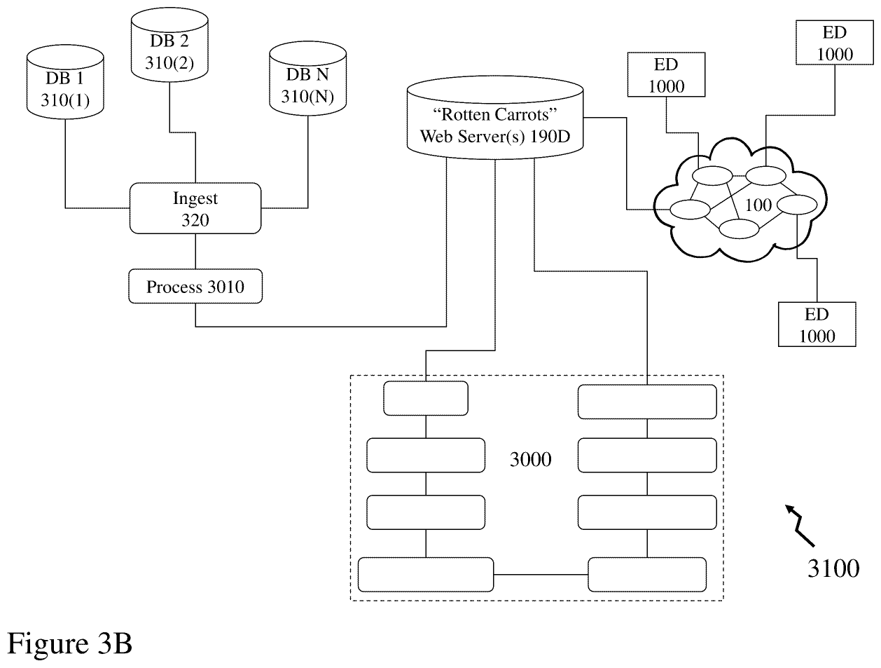

Also depicted in FIG. 1 are Electronic Devices (EDs) 100 according to embodiments of the invention such as described and depicted below in respect of FIGS. 3 to 6 and those described and depicted below. As depicted in FIG. 1 the EDs 100 communicate directly to the Network 100. The EDs 100 may communicate to the Network 100 through one or more wireless or wired interfaces included those, for example, selected from the group comprising IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, DSL, Dial-Up, DOCSIS, Ethernet, G.hn, ISDN, MoCA, PON, and Power line communication (PLC).

Accordingly, a user may exploit a PED and/or FED within an R-SAP 160, for example, and access one of the first or second primary content servers 190A and 190B respectively to perform an operation such as accessing/downloading an application which provides R-SAP features according to embodiments of the invention; execute an application already installed providing R-SAP features; execute a web based application providing R-SAP features; or access content. Similarly, a user may undertake such actions or others exploiting embodiments of the invention exploiting a PED or FED within first and second user groups 100A and 100B respectively via one of first and second cellular APs 195A and 195B respectively and first Wi-Fi nodes 110A. It would also be evident that a user may, via exploiting Network 100 communicate via telephone, fax, email, SMS, social media, etc.

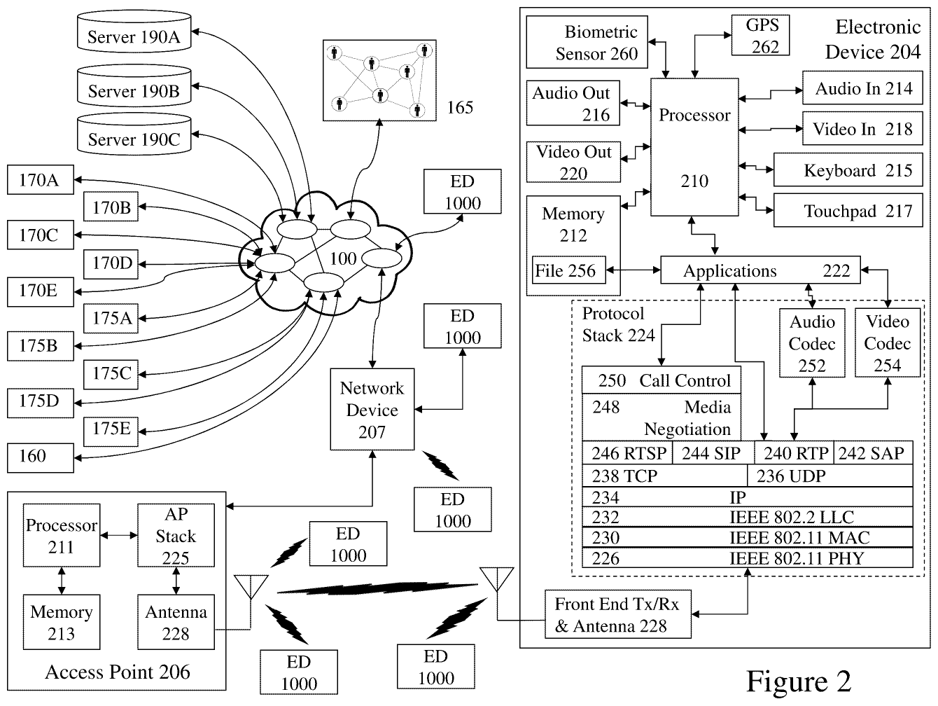

Now referring to FIG. 2 there is depicted an Electronic Device 204 and network access point 207 supporting R-SAP features according to embodiments of the invention. Electronic Device 204 may, for example, be a PED and/or FED and may include additional elements above and beyond those described and depicted. Also depicted within the Electronic Device 204 is the protocol architecture as part of a simplified functional diagram of a system 200 that includes an Electronic Device 204, such as a smartphone 155, an access point (AP) 206, such as first AP 110, and one or more network devices 207, such as communication servers, streaming media servers, and routers for example such as first and second servers 190A and 190B respectively. Network devices 207 may be coupled to AP 206 via any combination of networks, wired, wireless and/or optical communication links such as discussed above in respect of FIG. 1 as well as directly as indicated. Network devices 207 are coupled to Network 100 and therein Social Networks (SOCNETS) 165, first and second service providers 170A and 170B respectively, first and second third party service providers 170C and 170D respectively, a user 170E, first and second enterprises 175A and 175B respectively, first and second organizations 175C and 175D respectively, and a government entity 175E.

The Electronic Device 204 includes one or more processors 210 and a memory 212 coupled to processor(s) 210. AP 206 also includes one or more processors 211 and a memory 213 coupled to processor(s) 210. A non-exhaustive list of examples for any of processors 210 and 211 includes a central processing unit (CPU), a digital signal processor (DSP), a reduced instruction set computer (RISC), a complex instruction set computer (CISC) and the like. Furthermore, any of processors 210 and 211 may be part of application specific integrated circuits (ASICs) or may be a part of application specific standard products (ASSPs). A non-exhaustive list of examples for memories 212 and 213 includes any combination of the following semiconductor devices such as registers, latches, ROM, EEPROM, flash memory devices, non-volatile random access memory devices (NVRAM), SDRAM, DRAM, double data rate (DDR) memory devices, SRAM, universal serial bus (USB) removable memory, and the like.

Electronic Device 204 may include an audio input element 214, for example a microphone, and an audio output element 216, for example, a speaker, coupled to any of processors 210. Electronic Device 204 may include a video input element 218, for example, a video camera or camera, and a video output element 220, for example an LCD display, coupled to any of processors 210. Electronic Device 204 also includes a keyboard 215 and touchpad 217 which may for example be a physical keyboard and touchpad allowing the user to enter content or select functions within one of more applications 222. Alternatively, the keyboard 215 and touchpad 217 may be predetermined regions of a touch sensitive element forming part of the display within the Electronic Device 204. The one or more applications 222 that are typically stored in memory 212 and are executable by any combination of processors 210. Electronic Device 204 also includes Biometric Sensors 260, which acquire biometric data relating to the user passed to the Processor 210 and GPS 262, which provides geographical location information to passed to the Processor 210.

Electronic Device 204 includes a protocol stack 224 and AP 206 includes a communication stack 225. Within System 200 protocol stack 224 is shown as IEEE 802.11 protocol stack but alternatively may exploit other protocol stacks such as an Internet Engineering Task Force (IETF) multimedia protocol stack for example. Likewise, AP stack 225 exploits a protocol stack but is not expanded for clarity. Elements of protocol stack 224 and AP stack 225 may be implemented in any combination of software, firmware and/or hardware. Protocol stack 224 includes an IEEE 802.11-compatible PHY module 226 that is coupled to one or more Front-End Tx/Rx & Antenna 228, an IEEE 802.11-compatible MAC module 230 coupled to an IEEE 802.2-compatible LLC module 232. Protocol stack 224 includes a network layer IP module 234, a transport layer User Datagram Protocol (UDP) module 236 and a transport layer Transmission Control Protocol (TCP) module 238.

Protocol stack 224 also includes a session layer Real Time Transport Protocol (RTP) module 240, a Session Announcement Protocol (SAP) module 242, a Session Initiation Protocol (SIP) module 244 and a Real Time Streaming Protocol (RTSP) module 246. Protocol stack 224 includes a presentation layer media negotiation module 248, a call control module 250, one or more audio codecs 252 and one or more video codecs 254. Applications 222 may be able to create maintain and/or terminate communication sessions with any of devices 207 by way of AP 206. Typically, applications 222 may activate any of the SAP, SIP, RTSP, media negotiation and call control modules for that purpose. Typically, information may propagate from the SAP, SIP, RTSP, media negotiation and call control modules to PHY module 226 through TCP module 238, IP module 234, LLC module 232 and MAC module 230.

It would be apparent to one skilled in the art that elements of the Electronic Device 204 may also be implemented within the AP 206 including but not limited to one or more elements of the protocol stack 224, including for example an IEEE 802.11-compatible PHY module, an IEEE 802.11-compatible MAC module, and an IEEE 802.2-compatible LLC module 232. The AP 206 may additionally include a network layer IP module, a transport layer User Datagram Protocol (UDP) module and a transport layer Transmission Control Protocol (TCP) module as well as a session layer Real Time Transport Protocol (RTP) module, a Session Announcement Protocol (SAP) module, a Session Initiation Protocol (SIP) module and a Real Time Streaming Protocol (RTSP) module, media negotiation module, and a call control module. Portable and fixed electronic devices represented by Electronic Device 204 may include one or more additional wireless or wired interfaces in addition to the depicted IEEE 802.11 interface which may be selected from the group comprising IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, DSL, Dial-Up, DOCSIS, Ethernet, G.hn, ISDN, MoCA, PON, and Power line communication (PLC).

Also depicted in FIG. 2 are Electronic Devices (EDs) 100 according to embodiments of the invention such as described and depicted below in respect of FIGS. 3A to XXX. As depicted in FIG. 2 an EDs 100 may communicate directly to the Network 100. Other EDs 100 may communicate to the Network Device 207, Access Point 206, and Electronic Device 204. Some EDs 100 may communicate to other EDs 100 directly. Within FIG. 2 the EDs 100 coupled to the Network 100 and Network Device 207 communicate via wired interfaces. The EDs 100 coupled to the Access Point 206 and Electronic Device 204 communicate via wireless interfaces. Each ED 100 may communicate to another electronic device, e.g., Access Point 206, Electronic Device 204 and Network Device 207, or a network, e.g., Network 100. Each ED 100 may support one or more wireless or wired interfaces including those, for example, selected from the group comprising IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, DSL, Dial-Up, DOCSIS, Ethernet, G.hn, ISDN, MoCA, PON, and Power line communication (PLC).

Accordingly, FIG. 2 depicts an Electronic Device 204, e.g. a PED, wherein one or more parties including, but not limited to, a user, users, an enterprise, enterprises, third party provider, third party providers, wares provider, wares providers, financial registry, financial registries, financial provider, and financial providers may engage in one or more financial transactions relating to an activity including, but not limited to, e-business, P2P, C2B, B2B, C2C, B2G, C2G, P2D, and D2D via the Network 100 using the electronic device or within either the access point 206 or network device 207 wherein details of the transaction are then coupled to the Network 100 and stored within remote servers.

Optionally, rather than wired and/or wireless communication interfaces devices may exploit other communication interfaces such as optical communication interfaces and/or satellite communications interfaces. Optical communications interfaces may support Ethernet, Gigabit Ethernet, SONET, Synchronous Digital Hierarchy (SDH) etc.

Accordingly, a user may access and R-SAP according to an embodiment using an Electronic Device 204 which may, for example, be a PED, FED, or wearable device.

Accordingly, a user may access and R-SAP according to an embodiment using an Electronic Device 204 which may, for example, be a PED, FED, or wearable device, whilst they are associated with one or more ED 1000 which may, for example, be a sensor, a biometric tracker, a wearable device, or a wearable sensor.

### Health Information Based Communities and Knowledge Incentive Methods

Published health information provides a framework within which users, from medical professionals to the general public, make decisions with respect to a range of matters ranging from aspects of their lifestyle to treatment of medical conditions, pharmaceutical choices etc. However, whilst the Internet has made a wealth of information accessible to users it has also led to propagation of poor information, information overload, etc. For instance, users in the general public may be unaware that the website offering health advice is sponsored by a pharmaceutical company and seeks to push their products or that the advice is being given by someone with no medical training or background. For medical professionals, maintaining their recommendations in an up-to-date manner or counter-advising patients who have absorbed poor advice/knowledge can be time consuming. Events such as the COVID-19 pandemic have further highlighted these and other issues where recommendations change frequently, conflicting advice is provided online, physical physician visits severely limited, etc.

Within the following description reference to an R-SAP may refer to a single R-SAP, two or more R-SAPs which are associated with each other through a common provider, host, etc., two or more R-SAPs which have no association, etc.

Embodiments of the invention through an R-SAP provide a system and methods for the acquisition of recommendations from one or more “peer” level issuers of health and medical recommendations, for example, and provides one or more portals allowing medical professionals to maintain their knowledge up to date in an efficient manner and users to acquire recommendations in plain language. For example, the R-SAP may provide one portal to medical professionals and another to the general public with different features, algorithms, database links etc. or it may provide a single portal where a profile of the user defines the different features, algorithms, database links etc.

Embodiments of the invention through an R-SAP provide a system to provide a medical professional with the ability to direct patients to the R-SAP and monitor their subsequent access to it.

Embodiments of the invention through an R-SAP provide a system for medical certification authorities to track medical professional knowledge acquisition and establish time expiring ratings of current knowledge, etc.

Embodiments of the invention through an R-SAP provide a system exploiting artificial intelligence to establish the content provided to different users and adjust the content based upon a user's profile.

The COVID19 pandemic has unleashed a public hunger for trustworthy medical decision support and virtual health care services. Medical professionals and the general public are searching for up-to-date, balanced, trustworthy guidance (in many instances plain language guidance). Additionally; the medical and societal uncertainty around the COVID19 pandemic coupled with the Government instituted “lock-downs” etc. have magnified the public desire for trustworthy health recommendations, where concurrent restrictions relating to direct face-to-face contact between medical professionals and the general public have been limited pushing users to access this information from online resources.

These public health restrictions, fear of COVID transmission, and new government health care funding models have led to the rapid escalation of virtual care services. Telephone, video conferencing and other secure portal systems have emerged to improve access to care during the pandemic, but in the “trust in the provider” has persisted as a key ingredient for communication success. Physicians and other primary health care providers have been the traditional target for curated and published medical decision support/health recommendations. However, for them the issue is staying up to date, particularly as these recommendations may be long detailed documents and may be published by multiple independent sources. For example, a physician in Ottawa, Ontario should be up to date with provincial (state) guidelines, federal guidelines, and international guidelines together with sourcing recommendations from peer reviewed journals, etc.

Typically, an expert medical provider, e.g., a physician, doctor, nurse practitioner, etc. will during an in-person consultation (e.g., one schedule for a predetermined period of time such as 15 or 20 minutes for example), summarizes decision support or health recommendations to the patient in plain language. An experienced medical provider may present these messages to the patient in a customized or personalized fashion based on one or more patient profile(s). The provider, e.g., medical professional, also works with their patients to assess local relevance, local truth, inferences and acceptability and the patient and provider come to a decision on one or more care or lifestyle interventions. However, factors such as patients having multiple issues, physicians trying to limit discussions to one issue at a time, the provider having no direct means of assessing the patient's absorption of the information, and the provider having no current means to measure a patient's background knowledge of health information or recommendations, etc. can negatively impact the resulting decision(s) or outcome(s).

Further, traditionally, health recommendations have been developed only for national or regional use. However, as has become evident through COVID-19 recommendations are being made at finer granularity by health authorities at a city level. Some users may be on the boundary of two regions, countries, etc. and hence may need to consider other aspects and/or recommendations. Accordingly, an R-SAP according to one or more embodiments of the invention provides for automation, customization and localized patient and provider inferences and decision support/health recommendations for both patients and providers.

An R-SAP according to one or more embodiments of the invention may exploit artificial intelligence (AI) and machine learning (ML) to customize messages and communicate and deliver guidance to the user and provider. An R-SAP according to one or more embodiments of the invention may provide an interactive automated delivery system which promotes recall and transfer of information, but also highlights health information questions, and therefore, the R-SAP can also include an option for a virtual doctor consultation for the public and “continuing medical education” (CME) activity for providers.

An R-SAP according to one or more embodiments of the invention may provide an interactive web-based system to communicate curated trustworthy recommendations from around the world to the public and providers using one or more spring-board narratives and AI customized plain language messages for each user.

An R-SAP according to one or more embodiments of the invention may collect and present both provider and consumer ratings or inference comments for priority, feasibility, acceptability cost and fairness in order to support the decision or intention to implement. Consulting health professionals remain the final step and thus there is an option in the system to request a virtual doctor consult, or for a doctor to request official CME recognition.

An R-SAP according to one or more embodiments of the invention will exploit what is referred to as “cool media” which may use lower-definition media but demands interaction and involvement on the part of the viewer. The viewer is subliminally invited to perceive gaps in the content themselves and respond with comments that demonstrate preferences and local relevance inferences for the information. The viewer must be familiar with the content in order to understand and engage in the medium. Accordingly, for example, an R-SAP according to one or more embodiments of the invention may, with respect to the general public user, exploit cartoons, animations, jokes, exemplary scenarios, e-learning programs, phone conversations, comic books, etc.

Accordingly, an R-SAP according to one or more embodiments of the invention provides for:
Curating and appraising recommendations from around the world for public and provider engagement.
Creating plain language messages from published health recommendations;
Customization or personalization of the presentation order for the plain language messages (i.e., based on gender, age, education level, lifestyle, country of origin, etc.).
Collecting and analyzing data on local (public and provider) relevance and inferences related to recommendations, exploiting what the inventor refers to as FACE 2.0 (feasibility, acceptability, cost, and equity) methodologies through aspect including, but not limited to, priority, feasibility, acceptability, cost concerns, equity/fairness, and intent to use.
Presenting public and expert inferences, ratings, and comments on health recommendations on the R-SAP for public ratings, involvement, and comments.
Integration with a primary care clinical practice and software, including electronic medical record system and secure portal communication systems to enable two-way (patient-provider) communication.
Integration with professional oversight, educational institutes, government organizations etc.
Finally, capturing metadata on public and expert inferences and ratings will close the feedback loop and help build knowledge related to patient values and preferences and other implementation science.

Within embodiments of the invention the acquisition of public and expert inferences, rating and comments allows provisioning of a global feedback loop, as health recommendations for example can be acquired by a system according to an embodiment of the invention from any organization, enterprise, Governmental agency etc. globally, such that as ideas converge towards truth then these inferences will influence future health recommendations.

COVID19 has accelerated the speed of science and the speed of the development of health recommendations generally. It has also allowed guideline experts to begin to use high quality international publications locally, a new phenomenon that is supported by two emerging methods “adolopement”, see for example Schunemann/Pottie 2018 BMJ Open, and the FACE instrument, see for example Pottie et al. “GRADE Concept Paper 1: Validating the “F.A.C.E” instrument using stakeholder perceptions of feasibility, acceptability, cost, and equity in guideline implement” (J. Clinical Epidemiology, March 2021). Adolopement refers to adopted, adapting, or updating existing health recommendations using Grading of Recommendations, Assessment, Development and Evaluation (GRADE) evidence to the decision framework (BMJ 2016), it will not be the focus of this patent. The other method is the FACE instrument which was developed for guideline developers' pre-publication to help assess stakeholder implementation constructs to inform the publication of the guidelines. This patent introduces new “FACE 2.0” methods, which uses the existing FACE constructs to assess, and thus rate, the health recommendations post publication and exploits an interactive web platform for large scale public involvement/inferences/ratings/comments. As science speeds up, providers themselves need access to medical education for recommendations and public inferences, ratings, and comments to help with the implementation process and accordingly an R-SAP according to one or more embodiments of the invention provides an interactive platform for continuing medical education associated with the presented recommendations.

Accordingly, an R-SAP according to one or more embodiments of the invention supports providers as well as the general public. Providers themselves need the recommendations but they also need the FACE 2.0 inference, ratings, and comments to help them improve the implementation of these issued recommendations as well as improving subsequent recommendations. Typically, within the prior art a practitioner would have to go through the collection of this recommendation information on their own. However, an R-SAP according to one or more embodiments of the invention provides providers with springboard metadata to support both streamlining and personalization of the process.

Accordingly, an R-SAP according to one or more embodiments of the invention provides for identification of top recommendations relevant for patients where these are passed through a multidimensional preference mapping exercise with the public and providers to improve recall and transfer of information. Acquisition of initial subscription details will provide demographics and subscriber preferences ratings to inform the AI aspects of the R-SAP with the metadata required for the preference customization or personalization of messages. Subsequently user activities within the R-SAP will provide for refinement of this metadata as well as the establishment of new metadata.

An R-SAP according to one or more embodiments of the invention automates the FACE 2.0 approach to capture public and provider data that will permit future AI delivery customization. In order to ensure the appropriate compliance etc. the R-SAP according to one or more embodiments of the invention whilst providing information, recommendations and opportunities for researching it will direct the general public user to engage with their provider to make final decisions and implement recommendations, and thus it will provide access to virtual provider appointments (with evidence-based recommendation trained providers). At the same time medical professional provider subscribers will have access to accredited continuing medical education points if they wish to complete rec learning exercises and surveys

Referring to FIG. 3A there is depicted an exemplary process Flow 300 for onboarding recommendations within an R-SAP according to an embodiment of the invention. Flow 300 begins with first step 320 wherein recommendations are ingested/onboarded to the R-SAP from a plurality of recommendation repositories, depicted as DB 1 310(1), DB 2 310(2) through to DB N 310(N). The R-SAP may poll the plurality of recommendation repositories for new recommendations or recommendation updates or the plurality of recommendation repositories may push new recommendations or recommendation updates to the R-SAP. From first step 310 the R-SAP executes Process 3000 comprising second to ninth steps 330 to 395 respectively before pushing processed content to Web Server(s) 190D. Accordingly, EDs 1000 may access the processed content from the Web Server(s) 190D via the Network 100. Within embodiments of the invention the R-SAP may be accessed from a software application installed upon each ED 1000 or through a portal accessible through a web browser upon the ED 1000.

Process 3000 as depicted comprises second to ninth steps 330 to 395 respectively which comprise:
Second step 330 wherein the R-SAP processes each ingested recommendation with one or more AI processes and/or one or more ML processes.
Third step 340 wherein the R-SAP generates one or more narratives from the processed ingested recommendation. Optionally, the R-SAP may generate one narrative for medical professionals and another for general public users. Optionally, the R-SAP may generate a narrative solely for general public users in dependence upon a demographic set of a plurality of demographic sets, e.g., under 15, 17-23, 24-50, 50 plus. The plurality of demographic sets may be predefined within the R-SAP or they may be defined by a classification of the ingested recommendation wherein an AI process or ML process within second step 320 is a classification process. Optionally, the R-SAP may generate a narrative for each demographic set of the plurality of demographic sets.
Fourth step 350 wherein the R-SAP generates an “expert commentary” which is targeted to medical professionals using one or more AI processes and/or one or more ML processes. Optionally, the R-SAP may generate an expert commentary in dependence upon a demographic set of a plurality of demographic sets, e.g., doctors, nurses, tele-health workers, etc. The plurality of demographic sets may be predefined within the R-SAP or they may be defined by a classification of the ingested recommendation wherein an AI process or ML process within second step 320 is a classification process. Optionally, the R-SAP may generate a narrative for each demographic set of the plurality of demographic sets.
Fifth step 360 wherein the R-SAP generates a “plain language” recommendation or actionable message statement which is targeted to medical professionals using one or more AI processes and/or one or more ML processes. A plain language recommendation or actionable message being one that presents the high-level summary of the recommendation in language targeted to the general public user base. Optionally, the R-SAP may generate a plain language recommendation statement in dependence upon a demographic set of a plurality of demographic sets, which may include for example the user's literacy level. Accordingly, embodiments of the invention can customize or personalize the plain language recommendation of actionable statements to the health consumer literacy level wherein, for example, translated to language commensurate with a level of academic or mental progress such that these actionable statements are translated to language at that level. This may also include the use of symbols to make it easier for the user to read and understand, particularly for those with low literacy skills. The plurality of demographic sets may be predefined within the R-SAP or they may be defined by a classification of the ingested recommendation wherein an AI process or ML process within second step 320 is a classification process. Optionally, the R-SAP may generate a plain language recommendation for each demographic set of the plurality of demographic sets.
Sixth step 370 wherein the R-SAP generates “plain language” messaging to be presented to a user accessing the R-SAP where the messaging is, for example, a series of bullet points relating to the recommendation and/or the topic to which the recommendation relates. Optionally, the R-SAP may generate the messaging in dependence upon a demographic set of a plurality of demographic sets. The plurality of demographic sets may be predefined within the R-SAP or they may be defined by a classification of the ingested recommendation wherein an AI process or ML process within second step 320 is a classification process. Optionally, the R-SAP may generate messaging for each demographic set of the plurality of demographic sets, e.g., one for medical professionals, one for one general public demographic and another for another general public demographic. This customization or personalization may also be based on the Patient-Centered Clinical Method, see for example Stewart et al. “Patient Centered Medicine. Transforming the Clinical Method” (Sage Publications, Thousand Oaks, 1995). This well-regarded primary care clinical method uses patient's feelings, interest, function, and expectations to help tailor messages for the patient.
Seventh step 380 wherein the R-SAP generates local relevance or local truth questions to present to the user. A local truth may be viewed as a local relevance that has developed further to a social action, for example, and has reached a convergence of ideas relating to the recommendation(s).
These local relevance questions being used with respect to other AI processes and/or ML processes to assess user understanding, user decision making, etc. as well as feedback for refining the AI processes and/or ML processes employed in fourth to seventh steps 340 to 380, respectively. Optionally, the R-SAP may generate the local relevance questions in dependence upon a demographic set of a plurality of demographic sets. The plurality of demographic sets may be predefined within the R-SAP or they may be defined by a classification of the ingested recommendation wherein an AI process or ML process within second step 320 is a classification process. Optionally, the R-SAP may generate a local relevance questions for each demographic set of the plurality of demographic sets.
Eighth step 390 wherein the R-SAP generates metadata for storage upon a remote Web Server 190D in association with the results of the fourth to eighth steps 340 to 390, respectively. This metadata may include, for example but not be limited to, a mnemonic for the recommendation, a title of the recommendation, the source of the recommendation, a date and/or time of the recommendation being issued, an expiry of the recommendation (e.g. one established by the source of the recommendation or established by an AI process or ML process of the R-SAP), demographic data associated with the recommendation (e.g. for use in filtering recommendations for a user based upon their profile).
Ninth step 395 wherein the R-SAP posts the results of the fourth to eighth steps 340 to 390 respectively to the remote Web Server 190D which hosts an R-SAP, which within this specification is referred to as “Rotten Carrots.”

Within an embodiment of the invention Process 3000 is executed multiple times for a single ingestion event where each execution of the Process 3000 is associated with a demographic profile of a plurality of demographic profiles established, for example, by the R-SAP by default, in dependence upon metadata associated with the recommendation, in dependence upon the ingestion processing or a combination thereof.

Within embodiments of the invention, as described elsewhere within this specification, a user may obtain credits from public engagement with the recommendations which is fed back via the R-SAP to the user's professional association, electronic medical record (EMR) etc. Accordingly, through integration with the user's EMR those accessing the EMR, e.g., their doctor for example, can rapidly establish this history which uniquely communicates the user's, e.g., patient, background knowledge or experience with “plain language” recommendations.

Within the above description Process 3000 is described as a process executed once upon ingestion of a recommendation. However, referring to FIG. 3B there is depicted an exemplary process Flow 3100 for onboarding recommendations within an R-SAP according to an embodiment of the invention. Flow 3100 begins with first step Ingest 320 as does Flow 300 wherein recommendations are ingested/onboarded to the R-SAP from a plurality of recommendation repositories, depicted as DB 1 310(1), DB 2 310(2) through to DB N 310(N). The R-SAP may poll the plurality of recommendation repositories for new recommendations or recommendation updates or the plurality of recommendation repositories may push new recommendations or recommendation updates to the R-SAP. From first step 320 the Flow 3100 proceeds to second step 3020 wherein processing upon the ingested recommendation is undertaken wherein the results of the processing in second step 320 are stored upon the Web Server 190D. Subsequently, a user upon an ED 1000 performs an action, e.g., a search, which identifies the recommendation wherein the R-SAP executes Process 3000. Accordingly, Process 3000 executes once per user request relating to it but is processed using demographic and/or profile data of the user. In this manner, Process 3000 returns the information to the user such as narrative, “plain language” recommendation, messaging, local relevance questions, public comments, etc.

This information may be stored with metadata derived from the user's demographic and/or profile such that a subsequent request from a similar demographic or user profile is retrieved from the Web Server 190D rather than every request to the R-SAP resulting in execution of Process 3000 although this may be preferred in other embodiments of the invention.

One potential benefit of Flow 3100 over Flow 300 is that as the AI and/or ML processes evolve over time that the processing applied when the user requests is the most current.

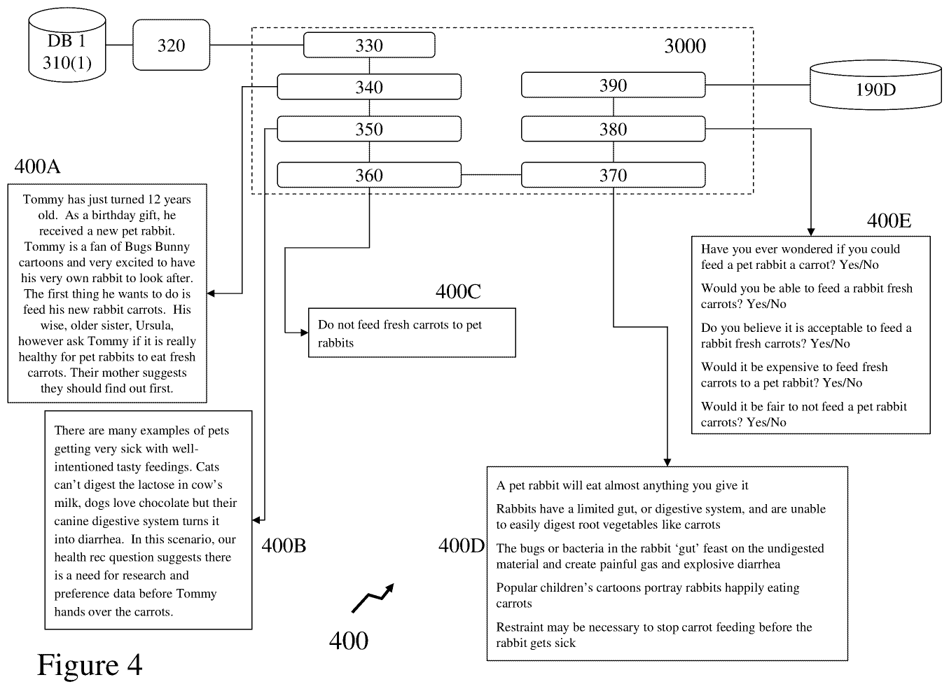

Now referring to FIGS. 4 to 6 respectively there are depicted exemplary recommendation ingestion and user directed outputs from a system according to an embodiment of the invention.

Accordingly, referring to FIG. 4 in Flow 400 a recommendation “Should I feed fresh carrots to my pet rabbit to keep him/her healthy?” Accordingly, the R-SAP executes Process 3000 resulting in first to fifth Boxes 400A to 400E respectively, these being the outputs from third to seventh steps 340 to 380 as described and depicted above in respect of FIG. 3A. The plain language recommendation is simply “Do not feed fresh carrots to pet rabbits.” Flow 400 may, for example, be targeted to a general public demographic where the local relevance questions allow the R-SAP to determine the user's understanding of the “plain language” recommendation, narrative etc. In some embodiments of the invention the R-SAP may adjust the narrative, plain language recommendation, messaging etc. to increase the user responses meeting a desired outcome. For example, in this example the recommendation is to a pet rabbit and adjusting the R-SAP outputs to increase the number of users taking the recommendation onboard is acceptable. However, in other instances the R-SAP does not adjust messaging etc. towards a desired outcome but to enhance user understanding and local relevance. Other questions may be presented to access this.

The local relevance question responses from users are processed by the R-SAP to generate local relevance ratings for a series of criteria. For example, these criteria may be priority, feasibility, acceptability, inexpensive and equity/fairness. Optionally, collected data may be analysed and employed within other applications outside those of the R-SAP directly such as providing Governmental organizations, local and international organizations, guideline developers, etc. with direct user feedback on their recommendations and/or policies.

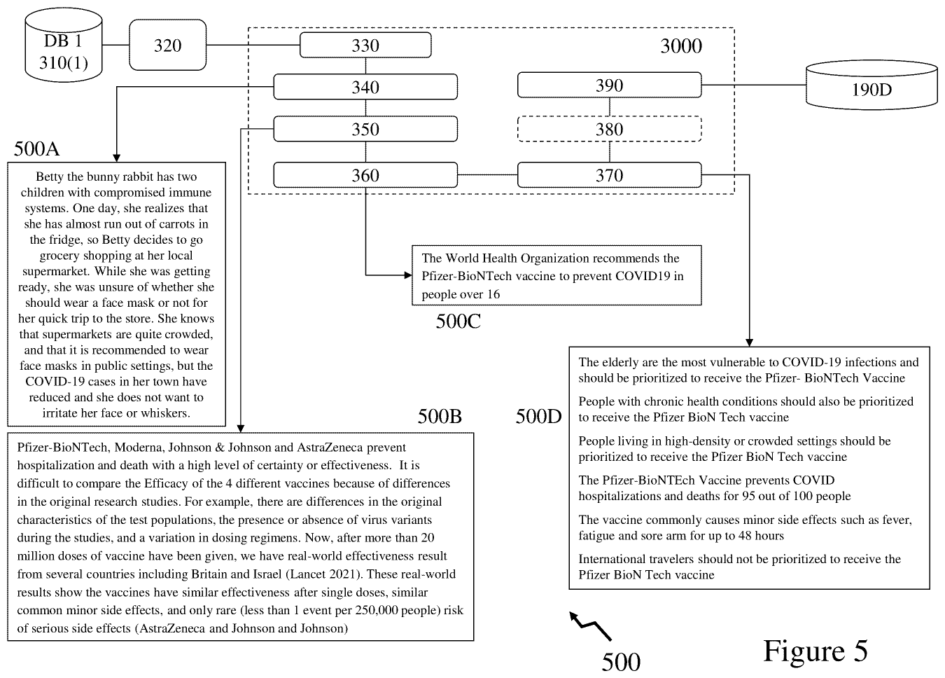

Now referring to FIG. 5 in Flow 500 a recommendation “Should people older than 16 years of age receive the Pfizer-BioNTech vaccine to prevent COVID-19?” Accordingly, the R-SAP executes Process 3000 resulting in first to fourth Boxes 500A to 500E respectively, these being the outputs from third to seventh steps 340 to 370 as described and depicted above in respect of FIG. 3A. The plain language recommendation is simply “The World Health Organization recommends the Pfizer-BioNTech vaccine to prevent COVID19 in people over 16.” In this instance the user demographic the R-SAP was generating for was medical professionals and accordingly in this instance the R-SAP determined that local relevance questions were not required.

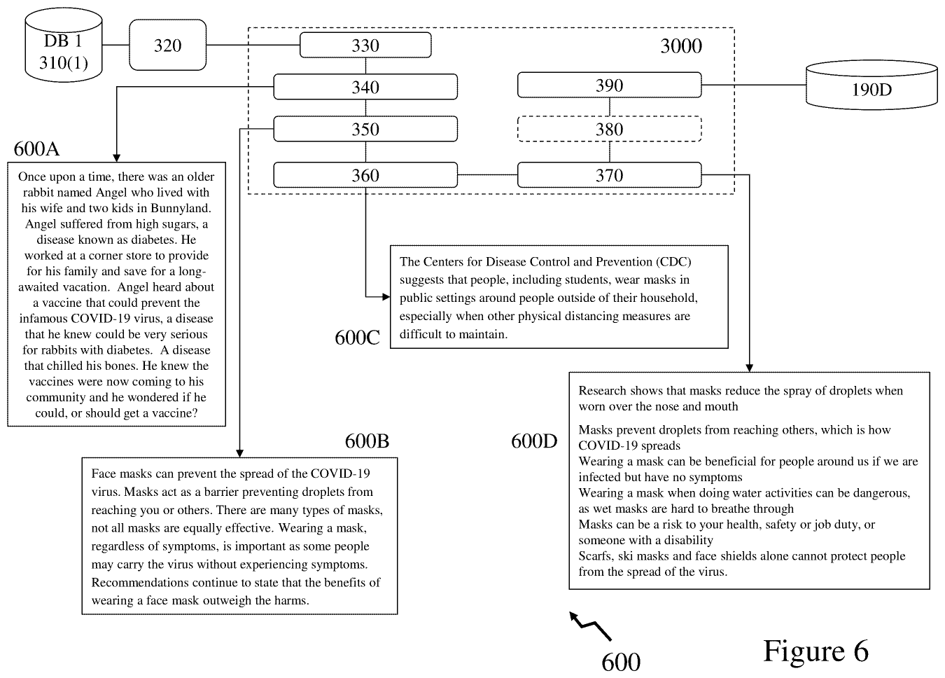

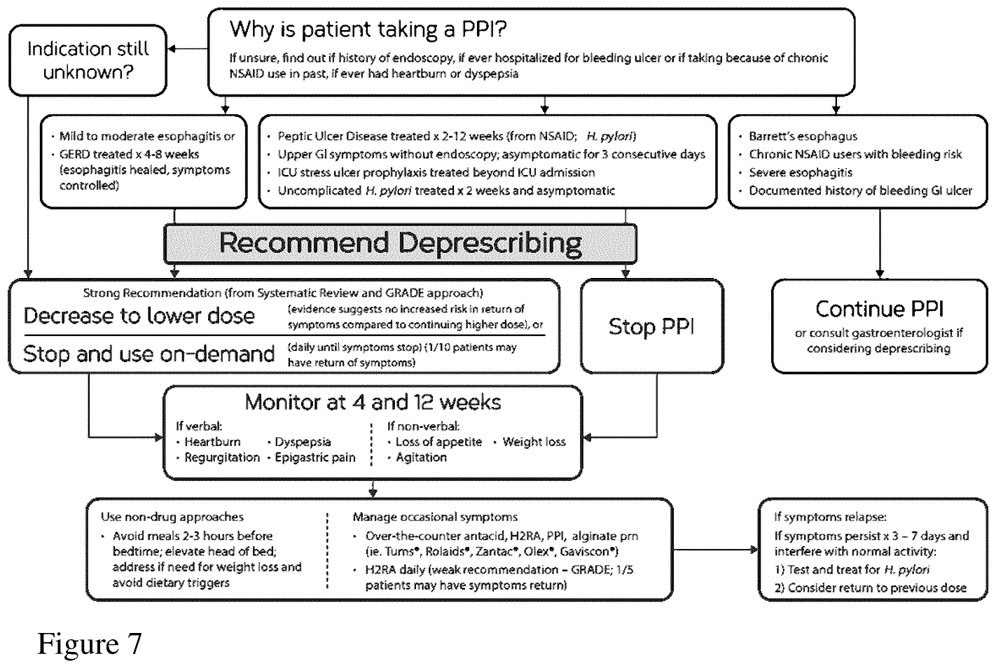

Referring to FIG. 6 in Flow 600 a recommendation “Should I wear face masks in crowded places to help prevent the spread of COVID-19?” Accordingly, the R-SAP executes Process 3000 resulting in first to fourth Boxes 600A to 600E respectively, these being the outputs from third to seventh steps 340 to 370 as described and depicted above in respect of FIG. 3A. The plain language recommendation is simply “The Centers for Disease Control and Prevention (CDC) suggests that people, including students, wear masks in public settings around people outside of their household, especially when other physical distancing measures are difficult to maintain.” In this instance the user demographic the R-SAP was generating for was a general public user in the United States. Accordingly in this instance the R-SAP determined that local relevance questions were not required but still potentially relevant for implementation and interventions, for example for vaccine hesitant populations.

Whilst the local relevance questions have been described and depicted with simple Yes/No responses it would be evident that the format of the questions and the answers may be any of those known in the prior art. Accordingly, these may be user selections from more options, require user input, be a rating, be based upon a sliding scale, including recording provider and public inferences and comments, etc.

Accordingly, an R-SAP, according to an embodiment of the invention allows a user to enter one or more search terms, keywords, etc. wherein the R-SAP identifies top recommendations relevant for the user and presents them to the user. The presented recommendations having been processed using one or more AI processes and/or one or more ML processes such as described and depicted above. The R-SAP enables a multidimensional preference mapping wherein local relevance questions/feedback is employed to improve recall and/or the transfer of information to the user. In many instances, demographic information, subscriber preferences, etc. will be acquired during a user's registration process with the R-SAP although others may be captured dynamically based upon user actions within the R-SAP. These demographic information, subscriber preferences, etc. are employed, for example, by the one or more AI processes and/or one or more ML processes in the customization of messaging presented to the user.

In order to make recommendations, which may be issued by international authorities or authorities within other jurisdictions to that the user resides within, locally useful and trustworthy, an R-SAP may exploit what the inventors refer to as the “FACE 2.0” approach to capture public and provider data that will permit future AI delivery customization.

An R-SAP once the recommendation(s) have been presented to the user and their feedback acquired the R-SAP will typically recommend that a general public user talk with their healthcare provider, e.g., a general practitioner, to make final decisions and implement recommendations. Within other embodiments of the invention the R-SAP may interface with the healthcare provider to establish a time/location for the subsequent consultation with respect to the recommendations. Optionally, the R-SAP may provide instant access to virtual healthcare provider appointments where the virtual healthcare providers are those having performed at least recommendation based training where the R-SAP tracks the virtual healthcare providers activities to establish the evidence of their “training.” Accordingly, the R-SAP may only offer appointments with virtual healthcare providers who have reviewed the recommendation(s) the user has reviewed in reaching the point of triggering the meeting.

This ability for the medical professional users to have their actions tracked in terms of tracking their engagement in learning exercises to learn new recommendations can be leveraged within professional related activities for the medical professional user. For example, reviewing a predetermined number of recently issued recommendations may acquire the user a predetermined period of time of professional accreditation time or be classed as accredited continuing medical education points. Within an embodiment of the invention the R-SAP may facilitate recommendation-based learning exercises and surveys.

As noted above an R-SAP may pull recommendations from external sources or they are pushed to the R-SAP by the external sources. However, this automated process may be augmented by a periodic, e.g., weekly, database search performed by personnel associated with the R-SAP provider. Optionally, the R-SAP may assign a priority setting to the recommendation established upon one or criteria, including, but not limited to, is relevant for patients and/or primary care, inclusion of guideline development method, inclusion of recommendation statements that refer to a population, intervention, comparison, and outcomes. A recommendation posted into the R-SAP could include a link to the published guideline reference for that recommendation allowing a user to access it if appropriate. This may form part of the metadata stored by the R-SAP with the processed recommendation.

As noted above an R-SAP generates plain language messages for users. Accordingly, within an embodiment of the invention the R-SAP executes a process comprising the steps of:
Obtaining the recommendation as input.
Scientifically appraising the recommendation, for example using AGREE II, an international tool to assess the quality and reporting of recommendations, to establish a recommendation development quality rating.
Extract key recommendation messages for users, both patients and/or providers, which relate to plain language messages derived from the recommendation. For example, these may include who is if for, benefits of intervention, harms of the intervention, strength of the recommendation, certainty of the evidence, other considerations, or implementation issues, etc.
Translate key recommendation messages for users, patients and/or providers in plain language or common man's language.

For example, the World Health Organization (WHO) recommendation on the Pfizer vaccine for COVID-19 when processed results in the plain language messages outlined below in Table 1.

TABLE 1
R-SAP Plain Language Messaging for
WHO Pfizer Vaccine Recommendation

Plain Language Messages
The Pfizer-BioNTech vaccine is 95% effective in preventing COVID-19.
The known benefits of the Pfizer-BioNTech vaccine are greater than the
known side effects.
The elderly should be prioritized to receive the Pfizer-BioNTech vaccine.
People with chronic health conditions should be prioritized to receive the
Pfizer-BioNTech vaccine.
People living in high density settings should be prioritized to receive the
Pfizer-BioNTech vaccine.
International travelers should not be prioritized to receive the Pfizer-
BioNTech vaccine.

Typically, this process may exploit one or more plain language editors within one or more AI processes and/or one of more ML processes with the final selection of a number of short messages, e.g., 5-7.

Once the plain languages messaging has been generated then one or more customizations may be applied exploiting using, for example, multidimensional preference ratings for each of the key recommendation messages. The R-SAP is configured to collect demographic data from site visitors, users (e.g., general public and provider (medical professional) subscribers). The R-SAP also collects from site visitors and users a rating of critical, important, not so important, and not important for each of the key recommendation messages. This data is then analyzed, for example statistically, with respect to different demographics characteristics, for example, age, gender, education level, country of origin, and health care provider or not health care provider in order to provide feedback to the plain language messaging generation engines within the R-SAP (each engine being for example one or more AI processes and/or one or more ML processes) to enhance the plain language generation for different demographics. Further, as new site visitors, public and provider subscribers arrive at the R-SAP it collects their demographics and use one or more engines (each engine being for example one or more AI processes and/or one or more ML processes) to link the incoming user with a preferred order of key recommendation messages. This may be within an initial graphical user interface (GUI) before the user enters any specific search terms etc. for recommendations relating to specific topics of interest to them.

The R-SAP will also collect multidimensional preference ratings from these new users to feed into the R-SAP analysis and AI/ML personalized message delivery processes.

With respect to presentation of expert and/or consumer local relevance then each site visitor, public or provider subscriber receives one or more questions related to the local or personal relevance of the recommendation for that user. For example, a complete series of FACE 2.0 questions may be presented to the user. An exemplary measurement scale for user responses may be a Likert scale. Exemplary questions could include:
What is the Priority?
What is the Feasibility?
What is the Acceptability?
What are the Out-of-Pocket Cost implications?
What is the fairness for their community?

Site visitors or site tourists will receive one or more questions which may be randomly selected so that with repeat visits the user does not default to previous answers. The R-SAP subsequently statistically synthesize this data along with the demographic data in order to subsequently present the results of this user local relevance or perspective data to subsequent incoming users. Visitors to an R-SAP according to an embodiment of the invention may also be invited to provide inference related comments to enhance the precision of the ratings. Optionally, within an embodiment of the invention only aggregated data may be presented to non-subscribers to the R-SAP but full local data is provided to full subscribers.

For example, with respect to the COVID-19 vaccine from Pfizer the recommendation from the World Health Organization is that is “recommends the new “Pfizer-BioNTech” vaccine to prevent COVID-19 for people aged 16 years and older.”

Accordingly, for a medical professional user the local relevance questions presented may include:
Is the recommendation topic a priority for you or your patients?
Is the recommendation feasible for you or you patients?
Is the recommendation acceptable for you or your patients?
Are the out-of-pocket costs/time commitment high for you or your patients?
Would the recommendation be fair for your community?

Then a final question is presented:
Do you intent to use or follow the recommendation?

For which an exemplary response scale is Yes/Probably yes/Probably no/No/Varies/Do not know.

An R-SAP according to an embodiment of the invention may present the public and provider local relevance ratings as well (again it may be only a composite for the nonsubscribers but more granular results would be available for the subscribers). The number of questions presented may vary for subscribers versus non-subscribers as may the granularity of the responses, e.g., non-subscribers reduced to 2 options whereas subscribers may have 4, 5, or 6 so that the AI/ML processes become more accurate for the subscriber. Again, within embodiments of the invention an R-SAP may facilitate users providing inference-related comments to enhance the precision of the ratings.

As noted above an R-SAP according to an embodiment of the invention may provide opportunity for medical professionals and other primary healthcare professionals to establish continuing medical education (CME) credits or education credits based upon point relating to each recommendation reviewed by the user. For example, the R-SAP may initially collect provider subscriber (e.g., medical professionals rather than general public) details and then offer these provider subscribers would then have the opportunity to choose if they would like to have preference rating, local relevance rating and reflection exercises from reviewing recommendations qualify for CME points. The R-SAP would have to have official accreditation from the relevant National Primary healthcare Provider Colleges for these CME activities. The National Primary Healthcare Provider Colleges would then receive data relating to the provider subscriber usage, either only those exceeding a CME credit threshold, or all provider subscribers to assess provider subscribers' overall maintenance of being cognizant of current recommendations even if they are not seeking CME credits. Optionally, the CME credits would be automatically sent to the provider upon successful completion, e.g., by email, text etc. for subsequent submission or to both in parallel.

Optionally, the National Primary healthcare Provider Colleges may advise provider subscribers of non-compliance with requirements if the provider subscribers fails to keep current on recommendations within their specific field(s).

Optionally, the National Primary healthcare Provider Colleges may publish a rating of provider subscribers based upon the statistics provided from the R-SAP in respect of the provider subscriber being current on recommendations. Optionally, the rating is the number of recommendations rather than being derived from it. Optionally, a recommendation within this scenario has a “lifetime” so that its counting towards their rating expires at the end of the lifetime so that a provider

As noted above an R-SAP according to an embodiment of the invention may provide a strategic link to a virtual doctor or to another primary healthcare professional via an appointment or other form of communication. Within an R-SAP according to an embodiment of the invention then an entry point to this is through a recommendation wherein, for example, at each step, for example, question, recommendation statement, preference rating, local relevance rating, and reflection the user is presented with a tab, button, icon or other interface within a GUI to make a virtual appointment with their doctor, a doctor or another primary health care provider. The user's personal doctor being established during the user's registration. Selecting the GUI element would take the user to an appointment request form which they complete and would be sent to the selected one of their doctor, another doctor, or another primary health care provider where the appointment request is logged into the user's profile or automatically communicated to the user's own doctor even if not with them. The appointment request form may include details of recommendations reviewed by the user within a predetermined period of time prior to triggering the appointment request, e.g., 15 minutes, this session, this and a prior session etc.

Automatically communicating to the user's own doctor may be implemented where the R-SAP is linked to the user's doctor's patient healthcare records so that when the doctor reviews the patient's file they are aware that the patient has been seeking other consultations rather than being unaware of their existence and can integrate this fact into their discussions with the patient. This data may be anonymized with respect to the identity of the other doctor or other primary healthcare provider.

The other doctor or another primary health care provider is established with a professional accredited to the R-SAP as a professional who is a Patient Centered Evidence Based Trained Primary Health Care Professional. Here integrating professional and general public users to a common platform can enhance service provisioning. Feedback from the user is also collected related to the virtual visit in order to further improve the system and remove those taking virtual visits etc. which yield consistently poor ratings or where those taking virtual visits are not maintaining their review of current recommendations.

Optionally, a primary care clinic may choose to register with the R-SAP where the clinic subscription would allow all registered patients to subscribe to the R-SAP and the “Virtual Care” as outlined in the preceding specification. Accordingly, where possible or as agreed to by all parties, the virtual care visits would be directed to the patient's clinic rather than other providers. Optionally, the R-SAP may be integrated within the electronic healthcare record (EHR) or electronic medical record (EMR) system(s) of the clinic in order to provide the provider subscribers of the client with easy access to the recommendations generally and during patient appointments specifically thereby enabling just in time physician decision support. According to the capabilities of the R-SAP, EHR/EMR system(s) etc. the clinic could provide customized newsletters and bulletins to its patients based upon issued recommendations on a regular basis. Accordingly, an R-SAP, according to an embodiment of the invention supports the digital first approach to educate before seeing a doctor.

Accordingly, an R-SAP may in addition to integrating other virtual visits into the EHR/EMR of the user for their doctor to see it may also, with the appropriate permissions, integrate a list of recommendations reviewed by the user with other metadata such as number of times the user has reviewed the recommendation, the data the user view the recommendation etc. In this manner the practitioner has a record of the recommendations viewed by their patient and can use this information within any subsequent consultations with the user.

Further, the user by virtue of viewing recommendations can gain points which at different thresholds of number completed trigger the issuance of a certificate as recognition of their engagement. Optionally, this user certificate may be redeemed through a service provider or retailer participating with the provider of the R-SAP so that users are encouraged to review recommendations. The redemption may be monetary, a credit, a discount or providing some product/service. Accordingly, the overall healthcare system can encourage users to keep up to date with recommendations relevant to them in a quick simple manner with benefits to the user and the healthcare system.

Accordingly, a user whilst employing embodiments of the invention may gain credits and/or a user certificate where the credits, for example, are established in dependence upon the recommendations viewed by the user. Accordingly, in dependence upon one or more factors such as, the number of credits, nature of the recommendations to which the credits are associated, a date and/or time of the recommendation being issued, an expiry of the recommendation; and demographic data associated with the recommendation may redeem, apply, or otherwise use the credits and/or a user certificate to obtain a benefit.

This benefit may, for example, be a financial reward, a financial discount, or a financial credit to employ in respect of purchasing at least one of a service and a product. Optionally, one or more thresholds may be established before the benefit is available to the user. Optionally, one or more thresholds may be established where upon exceeding a threshold the user is provided with an option with respect to at least one of a service and a product.

Within embodiments of the invention a user may access discretely or be directed to/invited to the R-SAP which is provided to them in the form of a website or a software application for example. User access of the R-SAP may be open access or subscriber based whilst user knowledge of the R-SAP may be established through different techniques including, for example, marketing, social media, crowdsourcing consumer networks, word-of-mouth etc. The R-SAP within embodiments of the invention can include a searchable and interactive application with emerging and existing health recommendations from local, national, international organizations which communicates these recommendations in a plain language format to the user. Such formats may include, for example, (i.e., AI driven marketing, cartoons, logos, plain “native” language at a user's educational level, audiovisual content, recommendation trailers etc. Accordingly, an R-SAP within embodiments of the invention allows consumers or other stakeholders (for example, patients, the general public, providers, purchases, payers, policy makers, product makers, principal investigators, and those engaged in Patient-Centered Outcomes (PCO) or PCO research (PCOR) framework(s) to view and review, with the potential in some embodiments of the invention for them to publicly rate the communicated and presented recommendations, best practice statements and other guidance. An R-SAP according to one or more embodiments of the invention may appear to be a media presentation platform to users but will embed learning and decision-making tips related to assessing the expert consensus and the public consensus and their related categories.

An R-SAP according to one or more embodiments of the invention may include a disclaimer statement that identifies information within the R-SAP as communicating health recommendations for the purposes of consumer and stakeholder education, and that all health recommendations, best practice statements and other guidance and even learning approaches to decision making should be discussed with, interpreted through or acted upon in collaboration with a clinician or health provider.

An R-SAP according to one or more embodiments of the invention may provide “users’ with search technology to identify health recommendations with expert and public consensus ratings. The R-SAP according to one or more embodiments of the invention allows for provisioning of a “plain language” summary to introduce the scientific recommendation or best practice statement development process (expert/critic rating consensus) thereby communicating the technical nuances of the development process and trustworthiness of recommendations for consumers, stakeholders, patients, and the public. By exploiting AI processes in conjunction with one or more user profile(s) the “plain language” can be tailored to the user, allowing for example for the “plain language” to reflect the user's demographic, educational level, history, etc.

Historically, recommendations have been developed almost exclusively as decision support for health providers and health policy consumers. In contrast consumer websites, consumer reports etc. provides ratings on health equipment but do not mention health recommendations nor are they transparent as to the ratings from other users etc. At present within the prior art detailed scientific methods to assess the development of guidelines and the trustworthiness of recommendations is being established with international consensus, such as for example, the Grading of Recommendations Assessment, Development and Evaluation (GRADE) working group, the Cochrane collaborative network, the World Health Organization (WHO), the “AGREE II” tool and the Guidelines International Network (GIN). At the same time, an internationally recognized GRADE Feasibility, Acceptability, Cost, and Equity (FACE) method has been reported that unlocks the potential patient and public stakeholder perspectives.

This complementary consumer or public perspective the inventor asserts can support the co-design of recommendation dissemination information and even the co-design of implementations themselves. For example, N. Chomsky describes within his publications the process of how media and the stories told by media can build various forms of a public consensus (see for example “Manufacturing Consent—The Political Economy of the Mass Media,” ISBN 0-375-71449-9). In contrast, the inventor through an R-SAP according to one or more embodiments of the invention exploits the R-SAP to build a consensus related to the decision-making process regarding emerging and existing recommendations.

An R-SAP according to one or more embodiments of the invention may present to the user expert consensus juxtaposed against public or consumer consensus for recommendations. The user is provided with technical details to demonstrate the expert consensus rating credibility, which may include, for example, a declaration of conflict of interest, scientific rigor and scope and purpose. The consumer details are established within the R-SAP according to one or more embodiments of the invention through mixed method surveys capturing ratings of priority, feasibility, acceptability, cost, equity, and intent to use.

Within an R-SAP according to one or more embodiments of the invention a user is invited to review recommendations together with associated critic and public ratings and to participate in establishing the public ratings to help advance consumer understanding of the recommendation or best practice statement.

An R-SAP according to one or more embodiments of the invention may also establish the consumer rating through obtaining other data from crowdsourcing consumer networks and social media. The user will discover a short (e.g., TICKR™) name for each recommendation, a graphic logo and a written plain language summary of the recommendation which may include, for example, a consumer video presentation (e.g., a trailer). The R-SAP user therefore seeks or discovers recommendations with plain language explanations, plain language critiques, public consensus ratings and qualitative consumer comments.

Within an R-SAP according to one or more embodiments of the invention new and/or emerging recommendations may be featured with expert consensus rating using, for example, a 1-100 scoring system with or without an associated five-star system. Accordingly, when these new or emerging recommendations enter the public domain, the R-SAP begins seeking and presenting public consensus data using one or more tools, e.g., the GRADE FACE Instrument (see for example K. Pottie et al. J. Clinical Epidemiology March 2021, pp. 133-140) discretely or in conjunction with one or more consumer networks, SOCNETs, and SOMEs for example. The R-SAP according to one or more embodiments of the invention may communicate to the user that the critics' consensus may change based on scientific feedback or updated published recommendations.

The user may elect to receive notifications of up-to-date expert ratings and public consensus ratings on emerging health recommendations. This may be particularly important for providers in the face of rapidly changing information, such as for example during the initial phases of the COVID-19 pandemic. An overall framework for an R-SAP according to one or more embodiments of the invention is to engage international guideline scientists and a large variety of consumers and public stakeholders, in the co-creation of implementation or “putting into use” stories. The “putting into use” may include, but is not limited to, provider information to improve communication in clinical practice, patient, and public comments for use as information in consumer networks, or as health information to be used for guidance for health policy makers. Further, the R-SAP according to one or more embodiments of the invention via linking this “putting into use” to the user's profile may increase the specificity/relevance of the “putting into use” data. For example, the AI driven R-SAP according to one or more embodiments of the invention may provide different “putting into use” data to a 50-year-old Asian female with respect to a recommendation than that it provides to a 21-year-old Caucasian male.

Accordingly, an R-SAP according to one or more embodiments of the invention provides web and software application tools that juxtaposes critics and public consensus ratings in order to communicate, engage and survey consumers on emerging and existing health recommendations, best practice statements and other guidance. The R-SAP according to one or more embodiments of the invention may include a clear disclaimer that the published recommendation and ratings are provided for information and education purposes only, and not as binding or official medical action or practice, but rather to communicate, reframe and engage consumers in information related to health recommendations, similar to consumer reports but based on health recommendations.

An R-SAP according to one or more embodiments of the invention may execute a process comprising the steps of ingesting a recommendation and processing the ingested recommendation with at least a plurality of artificial intelligence (AI) processes and a plurality of machine learning (ML) processes. The R-SAP may then generate a narrative from the processed ingested recommendation and an expert commentary targeted to medical professionals.

The R-SAP may then generate at least one of a plain language recommendation and a plain language actionable message statement targeted to the medical professionals before generating at least one of another plain language recommendation and another plain language actionable message statement targeted to non-medical professional users.

The R-SAP may then generate at least one of a local relevance question and a local truth question to present to each user accessing the ingested recommendation before generating metadata for storage within one or more web servers for use in generating and rendering to a user the ingested recommendation upon an electronic device associated with the user.

Within embodiments of the invention the R-SAP may also establish a plurality of clusters where each cluster of the plurality of clusters is established in dependence upon at least one of geographic locations of each user of a plurality of users accessing the system and profiles of each user of the plurality of users accessing the systems.

Accordingly, R-SAPs according to embodiments of the invention may then provide additional services and/or features to users of the R-SAP. For example, the R-SAP may manage documentation for each cluster of a plurality of clusters. The R-SAP may manage communications between users for each cluster of the plurality of users. The R-SAP may establish one or more knowledge-based communities for each cluster of the plurality of clusters.

As users of an R-SAP may be from different geographic locations their knowledge, inferences and influences will vary and accordingly the R-SAP manages these aspects such that a user's association to one or more clusters is based upon their geographic location information, for example extracted from their PED, FED, wearable device etc. used to access the R-SAP and/or their profile which is established by them upon registering with the R-SAP and augmented by additional information as they exploit the R-SAP or as added, with their permission, to their profile by a medical professional. Accordingly, a medical professional may tag the user's profile as “menopausal,” “cancer in remission,” “cancer,” etc. so that the user may be associated with a cluster of similar users so that they can view recommendations etc. as being viewed/provided to these other users in addition to viewing those they actively search for and/or identify.

Location information may be continuously acquired, periodically acquired or be constant until updated by the user. Optionally, the user may be able to temporarily tether their location such that, for example, their 4-week vacation to Paris, France from San Diego, California, U.S.A. does not result in the demographic set they are associated with changing during this period. Optionally, the tether may be a filtering such that only long-term shifts in location are applied to their profile where the filtering timeline is established by the R-SAP and/or user profile such that it is for example, a week, a month, 3 months, etc.

Further, the clustering concepts reflects that users in London, Ontario, Canada will typically have different knowledge, inferences and influences than another user in Beijing, China or one in Maseru, Lesotho. Accordingly, an R-SAP may establish each cluster to reflect the at least one of inferences, influences and knowledge of the user established from their profile and/or demographic analysis upon the user's profile.

An R-SAP according to embodiments of the invention may establish a narrative is a narrative of a plurality of narratives where each narrative is generated in dependence upon a demographic set of a plurality of demographic sets. The R-SAP may establish each demographic set of the plurality of demographic sets in dependence upon acquired location data relating to the user who is either a non-medical professional or a medical professional users. The R-SAP may allow the user to view content relating to other users within their demographic set of the plurality of demographics sets. Optionally, this content may be recommendations viewed by the other users within their demographic set of the plurality of demographics sets. Optionally, this content may be responses to local relevance questions presented to the other users within their demographic set of the plurality of demographics sets. Optionally, this content may be responses to local truth questions presented to the other users within their demographic set of the plurality of demographics sets.

An R-SAP according to an embodiment of the invention may in dependence upon establishing a demographic set provide the user associated with this demographic set with one or more other functions/features. Optionally, this function/feature may be the provisioning of a social media network/social media (SOCNET/SOME) for the demographic set of the plurality of demographic sets. This SOCNET/SOME may provide the users with messaging capabilities and other functions as known in the art for SOCNETS and/or SOMEs.

Optionally, this function/feature may be the provisioning of a virtual environment accessible to the user and other users within the demographic set of the plurality of demographic sets.

Optionally, these functions/features may be provisioned to the user and all other users without requiring their association with a cluster.

Whilst the following aspects with respect to questionnaires, quizzes and generating additional credits or scaling credits are presented with respect to a non-medical professional user it would be evident to one of skill in the art that the same aspects of embodiments of the invention may be applied to users accessing an R-SAP who are medical professionals.

As outlined above a user who is not a medical professional, i.e., a non-medical professional user, when accessing an R-SAP according to an embodiment of the invention is provided with access to plain language recommendations and plain language actionable message statement targeted to these non-medical professional users with respect to a recommendation issued upon one or more of a topic, disease, medical condition, health guideline, etc. which they either search for or the R-SAP highlights (i.e., recommends) to them based upon the user's previous interactions within the R-SAP and/or the user's profile. The inventor refers to these as recommendations “ingested” by the user. They may also be provided with at least one of a local relevance question and a local truth question which is presented to them either directly with the ingested recommendation (i.e. as part of the same GUI or GUIs as the ingested recommendation), upon the user reviewing the ingested recommendation and navigating away wherein the at least one of a local relevance question and a local truth question are presented to them within another GUI or GUIs, or upon the user selecting to answer the at least one of a local relevance question and a local truth question presented to them within another GUI or GUIs based upon their selection of an option within either the same GUI or GUIs as the ingested recommendation or a pop-up or GUI presented to them as they navigate away.

However, it would evident that within other embodiments of the invention that each of the local relevance question and the local truth question may be one of a series of questions presented to the user as part of a questionnaire or quiz (hereinafter referred to as a quiz for simplicity in the description). Within the description above “credits” may be generated in dependence upon the number of recommendations a user views. Within other embodiments of the invention the credit or credits for a recommendation may be modified, what the inventor refers to as a modified credit, where a modified credit is a scaling applied to the credit or credits for the recommendation such that the modified credit is employed in subsequent actions/activities with respect to the R-SAP (i.e. the modified credits replace the credit initially assigned to the recommendation). This scaling may within embodiments of the invention be a positive integer, a negative integer, a positive non-integer, a negative non-integer, and zero (i.e. the user gets no credit applied). The magnitude of the scaling may be established in dependence upon one or more factors including, but not limited to:
does the user answer a question correctly or not;
a degree of accuracy of the user's answer to the question where the question requires the user enter multiple elements in their response rather than a simple “yes or no (for example)” response;
how many questions the user answers;
how quickly the user answers each question;
is this a subsequent repetition of the quiz and adjusting the scaling based upon a degree of improvement/degradation in the accuracy of the responses from the user; and
does the user skip the quiz by default.

An answer to a question within a quiz presented to a user may include, but not be limited to:
selection of a positive or negative option rendered to the user;
selection of an option from a series of options rendered to the user; and
free format response from the user.

According to the capabilities of the electronic device upon which the user is accessing the R-SAP then a response to a question may be input to the electronic device by one or means including, but not limited to:
a keyboard input;
an input via a Braille keyboard;
an input via a haptic interface of the electronic device such as a mouse, touchscreen etc.;
an input via a microphone such as requiring speech or a sound (which may or may not included speech recognition upon the electronic device or remote to the electronic device by the R-SAP or another system accessible by the R-SAP and/or electronic device); and
an input via a camera with processing of the captured image or video to establish, for example, an action of the user (i.e. nodding their head, shaking their head, performing a predetermined action with their face (i.e., closing an eye, opening their mouth etc.)), and capturing sign language (which may or may not recognised upon the electronic device or remote to the electronic device by the R-SAP or another system accessible by the R-SAP and/or electronic device),

According to the capabilities of the electronic device upon which the user is accessing the R-SAP then a question rendered to the user via the electronic device by one or means including, but not limited to:
rendering as part of a GUI as text;
rendering as part of the GUI as sign language;
rendered as an audible signal (i.e. speech);
rendered upon a haptic interface forming part of or connected to the electronic device such as a Braille display for example.

Accordingly, as noted above the credits (i.e. the credits or modified credits where this feature is provided) are provided to the user to indicate their level of engagement/knowledge acquisition using the R-SAP but these may also be employed for other functions as identified above such as being employed in an accreditation activity of a user, as part of a continuing education requirement for the user, or as part of the user's electronic medical record for example. In the last instance, the non-medical professional user's medical record may include data dynamically linking to the R-SAP or provided by the R-SAP for example periodically (or upon each user's engagement with the R-SAP completing), but not be limited to, data to their engagement with the R-SAP such as the number of credits they have, a historical record of the credits they have to provide a temporal perspective of their engagement, the recommendations reviewed, and a breakdown of the recommendations reviewed and the credit earner for each recommendation reviewed.

Optionally, within other embodiments of the invention the quiz presented to the user may be a quiz generated independent of the R-SAP generating the plain language recommendations and plain language actionable messages or it may comprise a first portion automatically generated by the R-SAP when generating the plain language recommendations and plain language actionable messages and a second portion generated by one or more quiz generators which may be selected from the group comprising, one or more other non-medical professionals, one or more medical professionals, the R-SAP, one or more AI or ML systems associated with the R-SAP, and one or more AI and ML systems interfaced to the R-SAP.

### Asset Based Community Health Methods-“the Gardens”

Within the following section the inventors describe a conceptualization or visualization for Recommendation Systems, Applications and Platforms (R-SAPs) according to embodiments of the invention as described above. This conceptualization or visualization is referred to by the inventors as “The Garden” or “The Gardens” (hereinafter referred to as a garden) where a garden is associated with a user, a group of users, an enterprise, a service provider, a third party provider, a third party, a medical facility, one or more medical professionals, etc. However, it would be evident to one of skill in the art that these exemplary module outlines may be varied within other embodiments of the invention with respect to their content, structure, format, targeted medical issue, targeted health issue, etc. without departing from the scope of the invention.

The intention of a garden or set of gardens (“The Gardens”) is to provide users with community based primary care where the user (or users) access and exploit the underlying technology, artificial intelligence and ML/AI elements of the R-SAPs such that they feel organically and creatively acquiring knowledge which supports their health education and health development within the asset-based community health-based environment of “The Gardens” (hereinafter the inventors refer to this application as a Gardens R-SAP).

Each health consumer, i.e. user or patient, may be invited to register to a Gardens R-SAP which exploits location tracking, e.g. using GPS tracking, as discussed and outlined above. Each health consumer receives instructions with respect to the Gardens R-SAP and “digital real estate” to set up their own (digital) gardens to grow, reap and guide their health. Currently the medical-legal, physician controlled, electronic health records dominates the community care visit, and thus the medical home. This approach distorts the focus of community care and limits the discussion in provider visits, as it makes them medical-legal centric. Even the most dedicated community physicians/providers must fight to establish and keep the patient as the focus of care within a medical home framework.

The Gardens R-SAP represents a new concept in health education within a community environment leveraging today's social networking and technological era such that health consumer (i.e. a user or patient) has the opportunity to “build” their own garden within the Gardens R-SAP, i.e. build their own approach to health education. As the inventors view this analogy the health consumer through the Gardens R-SAP plant their own seeds for healthy behavioral changes, self-care, and guidance with respect to illness, medications, health knowledge etc. As such the Gardens R-SAP focusses on four pillars of community care: nutrition, movement (exercise), sleep and emotional health whilst offering electronic learning (e-learning) modules for the health consumer to build their own evidentiary support based upon these health pillars. The Gardens R-SAP according to embodiments of the invention documents personal ratings and reflections on their nutrition, movement, sleep and emotional health.

The Gardens R-SAP through other “gardens” also allows the health consumer to access family medical care, other health consumers, etc. either discretely or by becoming a “gardener” in one or more gardens wherein their experiences, knowledge are accessible to other gardeners who register to the same garden. For example, family medical care (e.g. the health consumer's personal family physician or other medical professional(s)) can provide through their garden within the Gardens R-SAP other e-learning resources and health resources for the health consumer to explore, rate and complete with the benefit of quizzes etc. as outlined according to embodiments of the invention above. The e-learning may, within embodiments of the invention, support the health consumer increasing their health learning level(s) by exploiting gamification software approaches to incentivize the health consumer.

A user's personal Garden may allow the user to include prescription medications, medical appointments, medical records, over-the-counter medications, alternative care medications, remedy lists, etc. The user's personal Garden may also integrate allergy information, records of acquired vital signs such as blood pressure, weight, oxygen saturation and others. Further, the user's Garden will also track and store personal social and knowledge assets of the user including family supports, languages spoken, cultural beliefs etc. . . . The user's personal Garden may also include a list, in lay terms, of ongoing health issues defined by the user. This user's Garden will also document and track the Garden's e-learning credits and certificates.

These records may be encrypted or otherwise secured so that they are solely accessible by the user or the user may enable others to access these records. For example, the user may grant access to one or more of their personal physician (e.g. family doctor), a medical professional, or a family member. Where the user grants their personal physician access then those aspects of the R-SAP described within this specification linking the user and their physician are enabled as the physician can see the user's e-learning activities as well as see what other medications, remedies, etc. the user is taking/using etc.

Accordingly, the personal Garden for a user becomes a first piece in the puzzle to link to primary care provider resource(s), e.g. the Gardens of the primary care provider(s), as well as other health related community resources, such as, but not limited to homecare, physiotherapy, etc. Accordingly, the user's personal Garden becomes a central access portal for the user so that that can access resources including, but not limited to, health information, health learning, primary health care, medical requirements and other community resources.

In addition to a user's personal Garden providing this shareable functionality, with patient permission, to the community physician/provider the Garden(s) support in-depth, proprietary, analytic software that summarises and presents a form of a “user weather report” (report or GUI dashboard) on the user's personal Garden to the community physician/patient to elevate the level of communication for in-person, virtual or digital care interactions. This “weather report” or summary will allow the provider and/or their physician to rapidly interpret not just their personal vitals, but also the user's educational, health literacy, mood, and social supports. This summary also provides insights into broader contributors that may impact the user's emotional health such as inclusion of community art exhibits, community events, community support, etc. For example, community and art exhibit participation may correlate to a consumer's emotional ability to see/visualize/actualize personal desires even in difficult times. The R-SAP software will support affirmation of a personal Garden including but not limited to, valuing a search for truth and beauty in life, with pluralism and non-monism supporting empowerment and health.

Within embodiments of the invention an R-SAP according to an embodiment of the invention may also incorporate a software module or modules that provide linkage to other software and/or features of a PED, FED, or mobile device associated with the user. For example, the R-SAP may automatically acquire and store biometric information or establish that a PED, FED, or mobile device should push biometric data to the R-SAP. Optionally, the R-SAP may track user's browser activities and/or filter electronic communications so that metadata or extracted information from these activities becomes part of the information stored within a user's personal Garden where the R-SAP employs filters, such as keyword filters, using defaults of the R-SAP and/or user defined terms. Accordingly, a user can re-access information through the R-SAP Garden portal originally accessed outside the R-SAP. This information may also aid a physician in their support of the user as they can see the user has been accessing homeopathic information selectively relative to pharmaceutical medications, for example, so that if the physician proposes a homeopathic approach to an issue it is more likely the user will adopt than if the physician recommends a pharmaceutical medication.

A “user weather report” may also communicate quiz results, personal recommendation ratings etc. based on previously described ratings on feasibility, acceptability, cost and fairness and intent to use. The “user weather report”, i.e. summary patient GUI dashboard, provides a limited number of targeted indicators to the user and/or physician so that they can rapidly absorb this snapshot of the user's health, issues, knowledge etc. Options within this dashboard may link to other metrics, other dashboards, GUIs, data etc. allowing the user and/or physician to obtain more detailed information on one or more aspects of the user.

This user weather report, may also, either using user inputs or analysis by one or more ML/AI processes include other data including, but not limited to, a communication or indication of readiness or non-readiness for engagement with respect to an issue, clues or indicators showing an ability to engage in or require deeper levels of conversation and/or guidance. Accordingly, the R-SAP and its user weather report allows for a patient and physician to resolve and address a critical step in any medical consultation/appointment as what is essentially a slow and awkward “blind-dating” process that currently dominates most healthcare visits can be bypassed as the physician can see the status, concerns, issues of the patient in the user weather report. Equally, issues a user may be hesitant to verbalize are easily communicated by the user's identification/searches/knowledge acquisition relating to this issue within the R-SAP which is highlighted within the user weather report.

On the community physician/provider side of the equation, the provider will be invited to do continuing medical education based on the same global leading evidence based guidelines and the providers will also receive ongoing e-learning training on how to interpret the ML/AI synthesised summary reports (user weather reports) from the patients (users). The R-SAP Gardens will be set up to collect data, categorise, and code to enable ML/AI within the garden communication system. Accordingly, the R-SAP according to embodiments of the invention supports regenerative economics where the building of health knowledge and beliefs and innovative communication leads to quantifiably better outcomes for the patients rather than just sustainable outcomes.

Within the following sections exemplary module outlines are presented with respect to three medical issues, de-prescribing proton pump inhibitors, de-prescribing benzodiazepines (commonly referred to as “Benzos”), and Diabetic Type 2 screening. These are presented to provide an overview of how a module may function within embodiments of the invention relating to Recommendation Systems, Applications and Platforms (R-SAPs) according to embodiments of the invention as described above. It would be evident to one of skill in the art that these exemplary module outlines may be varied within other embodiments of the invention with respect to their content, structure, format, targeted medical issue, targeted health issue, etc. without departing from the scope of the invention. Whilst the exemplary module outlines are with respect to three general medical issues it would be evident that other modules may be to general medical issues or they may be specific to a condition, symptom, treatment, drug, remedy etc.

### Exemplary Module Outline 1: De-Prescribing Proton Pump Inhibitors

Referring to FIG. 7 there is depicted an exemplary flow chart relating to determining whether a physician should recommend de-prescribing a proton pump inhibitor (PP) to a user. This flow chart being provided by the Bruyère Research Institute in Ottawa, Canada as part of a de-prescribing software application available for download by users, health care providers etc. The flow chart in FIG. 7 being derived from the work of Farrell et al. in “De-Prescribing Proton Pump Inhibitors, Evidence-based Clinical Practice Guideline” (Cam. Fam. Physician, Vol. 63, pp 354-364, 2017). Accordingly, it is evident that the flow is complex and not simple for a patient or physicians to follow.

Accordingly, an exemplary module within an R-SAP according to an embodiment of the invention may provide the user/physician with this information in an alternate manner enhancing its comprehension and application to the user. Upon accessing the module, either by searching for PPIs directly or through searching for a condition, such as acid reflux or gastroesophageal reflux disease (GERD), the user is initially presented with a short audiovisual introduction, e.g. an animation, video, etc. For example, the animation may be entitled “Bye Bye PPI” and sequence from an initial mountain of pills to a magician waving a wand and the mountain of pills reducing, disappearing or being replaced with exploding pills, exploding mountain of pills etc. The idea being to simply, quickly present the module's content in an audiovisual manner.

This is then followed by a series of GUIs that relate to a series of steps. For example, these may be Steps 1 to 5 as outlined below although the number of steps may be varied for other modules and their sequence may be varied or a user may bypass a step, go back to a step etc. These actions may be tracked such that the R-SAP indicates the user completed the module if they accessed very step and answered every question but reduces a measure of completion based upon skipping, not answering questions etc. The R-SAP may also track a user returning to the module multiple times and/or accessing a specific step/GUI so that this aspect is highlighted to a physician within a “user weather report” for example as being an aspect of particular interest/concern for the user. Within the following description exemplary steps, content and questions etc. are outlined and identified through italicised text to avoid the requirement for employing quotation marks in all instances so that non-italicised text relates to how the R-SAP operates etc. It would be evident to one of skill in the art that the text outlined below is for indication purposes and does not limit the content that may be presented to a user within an R-SAP according to an embodiment of the invention.

### Step 1: Educational Foundation

Engagement Question 1 “Did you know? You may be able to stop or reduce your antacid medication!” wherein the user responds through the GUI with yes or no (for example) and their response may be simply logged or be employed to skip part or all of this step.
   You may be able to say “Bye Bye” to your PPI.
   Why is this important? Continuous use of this medication may be harmful and unnecessary.
First, let's break it down.
   OK. Heartburn is common. A lot of people experience this from time to time. And that's normal. Eat a spicy meal. Have too much to eat. Drink too much coffee. And boom, your stomach will sometimes reward you with an overproduction of acid. Too much acid can cause a few things. First, if the acid stays in your stomach, we call that heartburn. That feeling of burning pain in your stomach or close to your lower rib cage or repeated belching or gas, yup that's heartburn for you!
   Next, if the acid climbs high into your esophagus. We call that esophagitis. An inflammation of your swallowing tube. This can sometimes cause chest pain, a feeling of food getting stuck at times or a feeling of pressure behind the sternum or middle chest. And if it travels higher, we call this reflux. People will sometimes get a bitter taste, acid taste or a sense of regurgitation which can contribute to a chronic cough, poor dentition, sore throat and even a hoarse voice at times, if it occurs regularly. When you suffer from heartburn, esophagitis or reflux more regularly we call this GERD (gastroesophageal reflux disease).
   So, heartburn that occurs once in a while is ok and not harmful. You can treat this with some simple over the counter medications like antacids when it happens. If you want to avoid medications all together, then try these simple changes in your diet and lifestyle to reduce chances of getting “burned”! Smaller meals, chewing your food, avoid late night eating, don't sleep with a tummy full of food, be careful of caffeine, coffee and alcohol as they too can contribute to excess acid in your stomach. Losing weight can also help reduce the pressure on your stomach contents.
   Many patients seek assistance from their healthcare provider when they do get these symptoms. Often, medications are prescribed. However, most patients may only need a short course of treatment and don't need to be on these medications for months or even years! And hence, there may be a way to say bye bye to your PPI!
Engagement Question 2 “Would you like to know more?” wherein the user responds through the GUI with yes or no (for example) and their response may be simply logged or be employed to skip part or all of this step. If the user selects to know more then other portions of the module and/or other modules may be accessed and rendered to the user.

### Step 2: Importance

“Did you know that if you've been taking an antacid pill for over 4 weeks that you may be eligible to stop this medication altogether?”
   Adults can often be diagnosed by their health care provider with heartburn (too much acid production), gastroesophageal reflux disease (acid back splashing into the esophagus) or even esophagitis (inflammation of the esophagus) which results in being prescribed medication (called a proton-pump inhibitor or PPI) to help relieve these annoying symptoms.
   Often, after 4 weeks of treatment, most of these conditions get better, however, patients and healthcare providers forget to discuss the ongoing use of these common medications
   Some patients may be able to consider stopping these medications safely and this can be done fairly easily over a period of time. Reducing unnecessary medications can actually help improve your overall health.
Engagement Question 3 “Is this important to you?” wherein the user responds through the GUI with, for example, free text entry or by selecting a defined option within a list of options and their response may be simply logged or be employed to define how subsequent portions of the module are presented to the user). Exemplary options in a list may include, for example, very important, important, not so important, and not important.

### Step 3: Feasibility/Acceptability

Did you know, studies now show that ongoing use of PPIs can cause rare, but harmful side effects such an increased risks of pneumonias, increased gastrointestinal infections, reduced absorption of vitamins (B12) and increased risk of fractures as you get older
   Sometimes, stopping an antacid that has been used for longer than 4 weeks can be difficult as the stomach reacts a bit differently without its helper and can overproduce acid temporarily. In this instance, reducing your PPI slowly over time may ease the stomach's ability to get off this medication effectively.
   To note, there are some individuals that need their PPI in order to protect the stomach from further damage. People that have had prior gastrointestinal bleeds, severe ulcers, have Barrett's esophagus or are needing anti-inflammatoires on a very regular basis, may need their PPIs!
   That is why it is important to speak to your healthcare provider about whether you, or a person you care for, really need to continue taking these medications
Engagement Question 4 “Is this practically achievable?” wherein the user responds through the GUI with yes or no (for example) and their response may be simply logged or be employed to skip part or all of this step.

### Step 4: Intent

To help you decide whether or not you are eligible to say bye bye to your PPI, we strongly suggest you speak to your healthcare provider the next time you see them
Engagement Question 5 “Are you ready for this?” wherein the user responds through the GUI with yes or no (for example) and their response may be simply logged or be employed to skip part or all of this step. Optionally, if the user replies “yes” then they may be subsequently asked if they wish to book an appointment with their healthcare provider wherein if they respond positively the R-SAP links them to a booking system for the healthcare provider or sends a text or other message to the healthcare provider.

### Step 5: Social Impact

PPI are quite common; in fact, they are often in the top 10 medications prescribed. Friends, family members or a person you care for, could consider deprescribing too!
Engagement Question 6 “Would you tell a friend about this?” wherein the user responds through the GUI with yes or no (for example) and their response may be simply logged or be employed to skip part or all of this step. If the user selects “yes” then they may be asked to enter a name of the friend wherein the R-SAP sends a message to the friend either through the R-SAP, to the Garden of the friend if they are registered (which may be indicated by the user), through a text or other electronic message which includes a link to this module of the R-SAP.

Within embodiments of the invention the R-SAP may through the user's physician authorising a de-prescription, either using physician input and/or one or more AI or ML processes, establish a de-prescribing schedule for the user as well other aspects such as providing reminders on when to reduce, generating prescription modifications for the de-prescription (e.g. moving to lower doses than current prescribed etc.) as well as prompting the user to respond to quizzes, questionnaires etc. to see if symptoms are appearing, being modified etc. as well as the user's perceptions of the de-prescription process.

Within this module the user may also be provided educational knowledge in a step such as being advised that certain foods and drinks may cause them heartburn such as fried foods, spicy foods, alcohol, coffee, citrus fruits or acidic foods such as tomatoes, oranges and salsa, or even garlic and onions. These may crank up their acid production at times where the user is then advised to eat less, so that smaller meals do not overwhelm their stomach and to avoid lying down for two hours.

With respect to a module targeted at medical professionals then the education aspects may, for example include indications and risks of long term use in bullet form for rapid absorption such as:
Appropriate Indications
   Prior gastrointestinal (GI) bleed
   Barrett's esophagus
   Los Angeles Grade D (severe) esophagitis
   Ongoing non-steroidal anti-inflammatory drug (NSAID) use
   Dual antiplatelet therapy (with prior upper gastrointestinal bleeding (UGIB), or one other risk factor)
   Ongoing hypersecretory conditions
Risks of Long Term Use
   Enteric infections (C. difficile, Campylobacter, Salmonella)
   Fractures⋅Pneumonia (hospital or community acquired)
   Spontaneous Bacterial Peritonitis in cirrhosis patients
   Hypomagnesemia
   Acute Interstitial Nephritis
   Vitamin B12 deficiency
The R-SAP may also provide additional information in extended form such as outlined below.
   When doing clinician education, make sure to include these team members, and outline the important role they can play in improving patient safety. Consider making two or four-week telephone follow-up a standard part of the intervention, provided by a non-physician team member.
   Don't maintain long term Proton Pump Inhibitor (PPI) therapy for gastrointestinal symptoms without an attempt to stop/reduce PPI at least once per year in most patients.
   PPIs are effective drugs for the treatment of gastro-esophageal reflux disease (GERD). Patients should always be prescribed the lowest dose of drug that manages their symptoms. Even though GERD is often a chronic condition, over time the disease may not require acid suppression and it is important that patients do not take drugs that are no longer necessary. For this reason patients should try stopping their acid suppressive therapy at least once per year. Patients with Barrett's esophagus, Los Angeles Grade D esophagitis, and gastrointestinal bleeding would be exempt from this.
   Don't maintain long-term Proton Pump Inhibitor (PPI) therapy for gastrointestinal symptoms without an attempt to stop/reduce PPI at least once per year in most patients.
   Canadian Association of Gastroenterology, Choosing Wisely Canada recommendation #1 Don't renew long-term proton pump inhibitor (PPI) therapy for gastrointestinal symptoms without an attempt to stop or reduce (taper) therapy at least once per year for most patients. Canadian Pharmacists Association Choosing Wisely Canada recommendation #4 Don't continue a proton pump inhibitor at discharge unless there is a compelling reason to continue therapy. Canadian Society of Hospital Pharmacists Choosing Wisely Canada recommendation #3
   This toolkit is well suited for your institution if you suspect that you have patients who are using PPIs on a continual basis and there has been no attempt to reduce their dosage or stop PPI use.

### Exemplary Module Outline 2: De-Prescribing Benzodiazepines (Benzos)

Accordingly, an exemplary module within an R-SAP according to an embodiment of the invention may provide the user/physician with this information in an alternate manner enhancing its comprehension and application to the user. Upon accessing the module, either by searching for benzos or sleep medications for example, the user is initially presented with a short audiovisual introduction, e.g. an animation, video, etc. For example, the animation may be entitled “Sleep meds get out of my head!” The idea being to simply, quickly present the module's content in an audiovisual manner.

This is then followed by a series of GUIs that relate to a series of steps. For example, these may be Steps 1 to 5 as outlined below although the number of steps may be varied for other modules and their sequence may be varied or a user may bypass a step, go back to a step etc. These actions may be tracked such that the R-SAP indicates the user completed the module if they accessed very step and answered every question but reduces a measure of completion based upon skipping, not answering questions etc. The R-SAP may also track a user returning to the module multiple times and/or accessing a specific step/GUI so that this aspect is highlighted to a physician within a “user weather report” for example as being an aspect of particular interest/concern for the user. Within the following description exemplary steps, content and questions etc. are outlined and identified through italicised text to avoid the requirement for employing quotation marks in all instances so that non-italicised text relates to how the R-SAP operates etc. It would be evident to one of skill in the art that the text outlined below is for indication purposes and does not limit the content that may be presented to a user within an R-SAP according to an embodiment of the invention.

### Step 1: Educational Foundation

Did you know that sleep meds can mess with your head?
You may be able to tell them, sleep meds get out of my head!
Engagement Question 1 “Would you like to know more?” wherein the user responds through the GUI with yes or no (for example) and their response may be simply logged or be employed to skip part or all of this step.
   Insomnia, or trouble sleeping, is one of the most common symptoms seen in primary care. It can present as a difficulty in initiating sleep or maintaining sleep which can start affecting our daytime functioning. Insomnia can be divided into a few quick categories, transient (<30 days), short term (<3 months) or chronic (>3 months). Transient or temporary insomnia is quite common and can present following an identifiable stressor or major life event such as stressful situations, exciting events, grief or loss, new shift work or a recent medical illness.
   Often, insomnia can be short lived and corrected with some simple lifestyle adjustments that help improve the ability and quality of your sleep. Practicing good sleep hygiene is key to achieving better sleep. Remember these simple steps: avoid caffeine, chocolate and alcohol around bedtime, avoid naps when possible during the day, sleep in a cool, dark, quiet environment, avoid stimulating activities before bed like exercise or video games, avoid using phone and computers screens before bed, allow yourself 30 minutes of relaxation time before sleep, if unable to sleep within 20-30 minutes, move to another room to sleep or get up to engage in a quiet activity then try again.
   Many patients seek assistance from their medical provider when they have trouble sleeping. At times, medications will be prescribed to help people with their insomnia. Different medications can be prescribed, such as benzodiazepines, Z-drugs and tricyclic antidepressants. Interestingly, patients will start taking them and often continue taking them for longer periods than required. But did you know that after 4 weeks of use, these medications become less efficient and somewhat problematic? Yes! They can actually mess with your head! And hence, there may be a way to say sleep meds get out of my head!
Engagement Question 2 “Would you like to know more?” wherein the user responds through the GUI with yes or no (for example) and their response may be simply logged or be employed to skip part or all of this step. If the user selects to know more then other portions of the module and/or other modules may be accessed and rendered to the user.

### Step 2: Importance

If you are over 18 years of age and have been using sleep medications for over 4 weeks, it is beneficial in considering reducing the use of these medications. If you are over 65, it is beneficial in considering avoiding the use of sleep medications as the risk of adverse side effects is even greater!
It is important to note that stopping these medications quickly can be more difficult. A more sensible approach is to reduce these slowly over a period of time. Developing a tapering plan is crucial in succeeding. Some may re-experience mild insomnia episodes or restlessness while reducing their doses, but done slowly and effectively, most can succeed. Those interested in stopping their sleeping medications see potential improvements in thinking and memory as well as obtaining a more natural sleep.
That is why it is important to speak to your healthcare provider about whether you, or a person you care for, really need to continue taking these sleep medications.
Engagement Question 3 “Is this important to you?” wherein the user responds through the GUI with, for example, free text entry or by selecting a defined option within a list of options and their response may be simply logged or be employed to define how subsequent portions of the module are presented to the user). Exemplary options in a list may include, for example, very important, important, not so important, and not important.

### Step 3: Feasibility/Acceptability

Did you know that sleep meds begin losing their efficacy after only 4 weeks! When medications, such as benzodiazepines are used for extended periods of time, the receptors in the brain physically change, making the medications less effective for sleep but contributing to increased drowsiness, balance issues, falls, cognitive problems, memory difficulties, slowing of daytime function and even increase the risk of motor vehicle accidents. Hence, sleep meds can mess with your head! More importantly, your natural sleep gets replaced with a chemical sleep and hence many patients can become physically dependent on these medications to initiate their sleep.
Often, patients and healthcare providers forget to discuss the ongoing use of these medications. Some patients may be able to consider stopping these medications safely and this can be done fairly easily over a period of time. Reducing meds that mess with your head can actually help improve your overall health.
Engagement Question 4 “Is this practically achievable?” wherein the user responds through the GUI with yes or no (for example) (for example) and their response may be simply logged or be employed to skip part or all of this step.

### Step 4: Intent

To help you decide whether or not you are eligible to say sleep meds get out of my head, we strongly suggest you speak to your healthcare provider the next time you see them.
Engagement Question 5 “Are you ready for this?” wherein the user responds through the GUI with yes or no (for example) and their response may be simply logged or be employed to skip part or all of this step. Optionally, if the user replies “yes” then they may be subsequently asked if they wish to book an appointment with their healthcare provider wherein if they respond positively the R-SAP links them to a booking system for the healthcare provider or sends a text or other message to the healthcare provider.

### Step 5: Social Impact

Sleeping medications are quite common, but they are considered problematic when used for more than 4 weeks. In fact, side effects become more concerning as we age. Friends, family members or a person you care for, could consider deprescribing too.
Engagement Question 6 “Would you tell a friend about this?” wherein the user responds through the GUI with yes or no (for example) and their response may be simply logged or be employed to skip part or all of this step. If the user selects “yes” then they may be asked to enter a name of the friend wherein the R-SAP sends a message to the friend either through the R-SAP, to the Garden of the friend if they are registered (which may be indicated by the user), through a text or other electronic message which includes a link to this module of the R-SAP.

### Exemplary Module Outline 3: Diabetic Type 2 Screening

Accordingly, an exemplary module within an R-SAP according to an embodiment of the invention may provide the user/physician with this information in an alternate manner enhancing its comprehension and application to the user. Upon accessing the module, either by searching for diabetes or diabetes symptoms, for example, the user is initially presented with a short audiovisual introduction, e.g. an animation, video, etc. For example, the animation may be entitled “Should I be Screened for Diabetes?” and sequence from an initial mountain of pills to a magician waving a wand and the mountain of pills reducing, disappearing or being replaced with exploding pills, exploding mountain of pills etc. The idea being to simply, quickly present the module's content in an audiovisual manner.

This is then followed by a series of GUIs that relate to a series of steps. For example, these may be Steps 1 to 5 as outlined below although the number of steps may be varied for other modules and their sequence may be varied or a user may bypass a step, go back to a step etc. These actions may be tracked such that the R-SAP indicates the user completed the module if they accessed very step and answered every question but reduces a measure of completion based upon skipping, not answering questions etc. The R-SAP may also track a user returning to the module multiple times and/or accessing a specific step/GUI so that this aspect is highlighted to a physician within a “user weather report” for example as being an aspect of particular interest/concern for the user. Within the following description exemplary steps, content and questions etc. are outlined and identified through italicised text to avoid the requirement for employing quotation marks in all instances so that non-italicised text relates to how the R-SAP operates etc. It would be evident to one of skill in the art that the text outlined below is for indication purposes and does not limit the content that may be presented to a user within an R-SAP according to an embodiment of the invention.

### Step 1: Educational Foundation

Screening and intervention for certain common conditions, like type 2 diabetes, can be extremely helpful especially if detected early and linked to education and lifestyle changes. Early awareness and or detection can allow us the opportunity to make small, easy changes that can help avoid further progression of the disease and even prevent more serious complications down the road.
Type 2 diabetes is one of those common conditions that can often develop slowly over the years without many experiencing many symptoms. How do you know if you are at risk of developing type 2 diabetes? How do you even know if you have type 2 diabetes?
Well, following simple early screening guidelines can help . . .
Engagement Question 1. “Would you like to know more?” wherein the user responds through the GUI with yes or no (for example) and their response may be simply logged or be employed to skip part or all of this step. If the user selects to know more then other portions of the module and/or other modules may be accessed and rendered to the user. For example, this may include:
So, what exactly is type 2 diabetes?
Well, let's begin by following the food we eat . . .
When we eat, our bodies start producing hormones that help us store the energy contained in food for immediate or future use. Insulin, produced by our pancreas, helps glucose enter our organs to be utilized as energy. Excess glucose or energy is then converted into storage or our fat cells. Type 2 diabetes develops when our pancreas gets really tired of producing insulin over the years. If we eat too frequently or eat more than our bodies require, our pancreas starts working overtime. These extra hours and extra shifts tires the pancreas out and makes our insulin either less effective over time or we start producing less insulin altogether. When insulin ceases to work efficiently or when we don't produce enough insulin, glucose stays in our pipelines and can't be transported to the organs that need it most. Hence, we get higher sugars in our blood stream. These high sugars over time, start causing damage or inflammation in our blood vessels. Many organs can therefore be affected by these changes, our hearts, our brains, our kidneys, our eyes. Hence why diabetes is a major risk factor for strokes, heart attacks, kidney failure, blindness and amputations.
So, if sugars can be elevated in our blood stream without causing many immediate symptoms, how do we know what our blood is doing?
Well, first, determining whether you are at a low or high risk of developing diabetes may help.
Engagement Question 2 “Would you like to know more?: wherein the user responds through the GUI with yes or no (for example) and their response may be simply logged or be employed to skip part or all of this step. If the user selects to know more then other portions of the module and/or other modules may be accessed and rendered to the user. For example, this may include:
If you are at low risk of developing diabetes, is it not recommended to screen yourself routinely with a blood test. Higher risk individuals may want to be screened every 3-5 years to ensure they keep track of their sugar status. Very high risk individuals should be screened early, as early detection can make a significant impact on their health!
A simple risk assessment tool can provide you enough information to help determine whether or not to be screened.
Engagement Question 3 “Is this important to you? wherein the user responds through the GUI with, for example, free text entry or by selecting a defined option within a list of options and their response may be simply logged or be employed to define how subsequent portions of the module are presented to the user). Exemplary options in a list may include, for example, very important, important, not so important, and not important.

### Step 3: Feasibility/Acceptability

The user may be presented with the results of one or more discrete screening tests or the results of an ongoing monitoring. The ML/AI processes of the R-SAP may analyse these one or more discrete screening tests and/or ongoing monitoring and present the user with a summary which might be in the form a simple colour, score etc. If the ML/AI processes determine the user is at risk then the R-SAP may present the user with a message such as:
Considering your scores on this risk calculator, you may want to speak with your healthcare provider about screening.
Engagement Question 4 “Is this practically achievable?” wherein the user responds through the GUI with yes or no (for example) and their response may be simply logged or be employed to skip part or all of this step. Alternatively, if the user answers yes then the user may be subsequently asked if they wish to book an appointment with their healthcare provider wherein if they respond positively the R-SAP links them to a booking system for the healthcare provider or sends a text or other message to the healthcare provider.

### Step 4: Intent

Consider reviewing this with your healthcare provider the next time you book an appointment to ensure you are properly screened with a simple blood test called Hemoglobin Alc.
Engagement Question 5 “Are you ready for this? wherein the user responds through the GUI with yes or no (for example) and their response may be simply logged or be employed to skip part or all of this step. Optionally, if the user replies “yes” then they may be subsequently asked if they wish to book an appointment with a clinic for the blood test wherein if they respond positively the R-SAP links them to a booking system for the clinic or sends a text or other message to their healthcare provider to provide them and/or a clinic with the requisite form for the blood test.

### Step 5: Social Impact

Type 2 diabetes is extremely common, in fact, many individuals may have type 2 diabetes and not even know it. Friends, family members or a person you care for, could consider screening too!
Engagement Question 6 “Would you tell a friend about this? wherein the user responds through the GUI with yes or no (for example) and their response may be simply logged or be employed to skip part or all of this step. If the user selects “yes” then they may be asked to enter a name of the friend wherein the R-SAP sends a message to the friend either through the R-SAP, to the Garden of the friend if they are registered (which may be indicated by the user), through a text or other electronic message which includes a link to this module of the R-SAP.

An R-SAP according to embodiments of the invention where acquiring biometric data of the user may automatically trigger a recommendation for them to review this module if the biometric data indicates an increased likelihood or risk of diabetes. Where a user is considered at risk but a blood test does not indicate

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps, and means described above may be done in various ways. For example, these techniques, blocks, steps, and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above and/or a combination thereof.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages and/or any combination thereof. When implemented in software, firmware, middleware, scripting language and/or microcode, the program code or code segments to perform the necessary tasks may be stored in a machine readable medium, such as a storage medium. A code segment or machine-executable instruction may represent a procedure, a function, a subprogram, a program, a routine, a subroutine, a module, a software package, a script, a class, or any combination of instructions, data structures and/or program statements. A code segment may be coupled to another code segment or a hardware circuit by passing and/or receiving information, data, arguments, parameters and/or memory content. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor and may vary in implementation where the memory is employed in storing software codes for subsequent execution to that when the memory is employed in executing the software codes. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels and/or various other mediums capable of storing, containing or carrying instruction(s) and/or data.

The methodologies described herein are, in one or more embodiments, performable by a machine which includes one or more processors that accept code segments containing instructions. For any of the methods described herein, when the instructions are executed by the machine, the machine performs the method. Any machine capable of executing a set of instructions (sequential or otherwise) that specify actions to be taken by that machine are included. Thus, a typical machine may be exemplified by a typical processing system that includes one or more processors. Each processor may include one or more of a CPU, a graphics-processing unit, and a programmable DSP unit. The processing system further may include a memory subsystem including main RAM and/or a static RAM, and/or ROM. A bus subsystem may be included for communicating between the components. If the processing system requires a display, such a display may be included, e.g., a liquid crystal display (LCD). If manual data entry is required, the processing system also includes an input device such as one or more of an alphanumeric input unit such as a keyboard, a pointing control device such as a mouse, and so forth.

The memory includes machine-readable code segments (e.g., software or software code) including instructions for performing, when executed by the processing system, one of more of the methods described herein. The software may reside entirely in the memory, or may also reside, completely or at least partially, within the RAM and/or within the processor during execution thereof by the computer system. Thus, the memory and the processor also constitute a system comprising machine-readable code.

In alternative embodiments, the machine operates as a standalone device or may be connected, e.g., networked to other machines, in a networked deployment, the machine may operate in the capacity of a server or a client machine in server-client network environment, or as a peer machine in a peer-to-peer or distributed network environment. The machine may be, for example, a computer, a server, a cluster of servers, a cluster of computers, a web appliance, a distributed computing environment, a cloud computing environment, or any machine capable of executing a set of instructions (sequential or otherwise) that specify actions to be taken by that machine. The term “machine” may also be taken to include any collection of machines that individually or jointly execute a set (or multiple sets) of instructions to perform any one or more of the methodologies discussed herein.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. A system comprising:
a memory accessible to one or more microprocessors storing a database comprising a plurality of recommendations;
another memory accessible to the one or more microprocessors storing another database comprising user data relating to a set of users;
a further memory accessible to the one or more microprocessors storing computer executable instructions which when executed by the one or more microprocessors configure the one or more microprocessors to execute a process comprising:
ingest a recommendation;
process the ingested recommendation with at least a plurality of artificial intelligence (AI) processes and a plurality of machine learning (ML) processes;
generate a narrative from the processed ingested recommendation;
generate an expert commentary targeted to medical professionals;
generate at least one of a plain language recommendation and a plain language actionable message statement targeted to the medical professionals;
generate at least one of another plain language recommendation and another plain language actionable message statement targeted to non-medical professional users;
generate at least one of a local relevance question and a local truth question to present to each user accessing the ingested recommendation;
generate metadata for storage within one or more web servers for use in generating and rendering to a user the ingested recommendation upon an electronic device associated with the user.

2. The system according to claim 1, wherein
the narrative is a narrative of a plurality of narratives; and
each narrative is generated in dependence upon a demographic set of a plurality of demographic sets.

3. The system according to claim 1, wherein
the narrative is a narrative of a plurality of narratives;
each narrative is generated in dependence upon a demographic set of a plurality of demographic sets; and
the plurality of demographic sets are defined by a classification of the ingested recommendation wherein at least one of an AI process of the plurality of AI processes and a ML process of the plurality of ML processes is a classification process.

4. The system according to claim 1, wherein
the narrative is a narrative of a plurality of narratives;
each narrative is generated in dependence upon a demographic set of a plurality of demographic sets; and
the demographic set of the plurality of demographic sets is established in dependence upon acquired location data relating to the user who is either a non-medical professional or a medical professional users.

5. The system according to claim 1, wherein
the narrative is a narrative of a plurality of narratives;
each narrative is generated in dependence upon a demographic set of a plurality of demographic sets;
the demographic set of the plurality of demographic sets is established in dependence upon acquired location data relating to the user who is either a non-medical professional or a medical professional users;
the user can view content relating to other users within their demographic set of the plurality of demographics sets; and
the content comprises at least one of:
recommendations viewed by the other users within their demographic set of the plurality of demographics sets;
responses to local relevance questions presented to the other users within their demographic set of the plurality of demographics sets; and
responses to local truth questions presented to the other users within their demographic set of the plurality of demographics sets.

6. The system according to claim 1, wherein
the narrative is a narrative of a plurality of narratives;
each narrative is generated in dependence upon a demographic set of a plurality of demographic sets;
the demographic set of the plurality of demographic sets is established in dependence upon acquired location data relating to the user who is either a non-medical professional or a medical professional users; and
the computer executable instructions further configure the one or more microprocessors to establish at least one of:
a social media network for the demographic set of the plurality of demographic sets; and
a virtual environment accessible to the user and other users within the demographic set of the plurality of demographic sets; and
a virtual environment accessible to the user and other users.

7. The system according to claim 1, wherein
the expert commentary is an expert commentary of a plurality of expert commentaries; and
each expert commentary is generated in dependence upon a demographic set of a plurality of demographic sets of medical professionals.

8. The system according to claim 1, wherein
the expert commentary is an expert commentary of a plurality of expert commentaries;
each expert commentary is generated in dependence upon a demographic set of a plurality of demographic sets of medical professionals; and
the plurality of demographic sets are defined by a classification of the ingested recommendation wherein at least one of an AI process of the plurality of AI processes and a ML process of the plurality of ML processes is a classification process.

9. The system according to claim 1, wherein
the at least one of a plain language recommendation and a plain language actionable message statement is one of a plurality of plain language statements; and
each plain language statement is generated in dependence upon a demographic set of a plurality of demographic sets of the medical professionals.

10. The system according to claim 1, wherein
the at least one of a plain language recommendation and a plain language actionable message statement is one of a plurality of plain language statements; and
each plain language statement is generated in dependence upon a demographic set of a plurality of demographic sets of the medical professionals; and
the plurality of demographic sets are defined by a classification of the ingested recommendation wherein at least one of an AI process of the plurality of AI processes and a ML process of the plurality of ML processes is a classification process.

11. The system according to claim 1, wherein
the at least one of the other plain language recommendation and the other plain language actionable message statement is one of a plurality of other plain language statements; and
each other plain language statement is generated in dependence upon a demographic set of a plurality of demographic sets of the other users.

12. The system according to claim 1, wherein
the at least one of the other plain language recommendation and the other plain language actionable message statement is one of a plurality of other plain language statements; and
each other plain language statement is generated in dependence upon a demographic set of a plurality of demographic sets of the other users; and
the plurality of demographic sets are defined by a classification of the ingested recommendation wherein at least one of an AI process of the plurality of AI processes and a ML process of the plurality of ML processes is a classification process.

13. The system according to claim 1, wherein
the at least one of a local relevance question and a local truth question is a query of a plurality of queries; and
each query is generated in dependence upon a demographic set of a plurality of demographic sets of users.

14. The system according to claim 1, wherein
the at least one of the local relevance question and the local truth question is a query of a plurality of queries; and
each query is generated in dependence upon a demographic set of a plurality of demographic sets of users; and
the plurality of demographic sets are defined by a classification of the ingested recommendation wherein at least one of an AI process of the plurality of AI processes and a ML process of the plurality of ML processes is a classification process.

15. The system according to claim 1, wherein
the metadata associated with an ingested recommendation comprises one or more elements selected from the group comprising:
a mnemonic for the recommendation;
a title of the recommendation;
the source of the recommendation;
a date and/or time of the recommendation being issued;
an expiry of the recommendation; and
demographic data associated with the recommendation.

16. A method comprising:
ingesting a recommendation;
processing the ingested recommendation with at least a plurality of artificial intelligence (AI) processes and a plurality of machine learning (ML) processes;
generating a narrative from the processed ingested recommendation;
generating an expert commentary targeted to medical professionals;
generating at least one of a plain language recommendation and a plain language actionable message statement targeted to the medical professionals;
generating at least one of another plain language recommendation and another plain language actionable message statement targeted to non-medical professional users;
generating at least one of a local relevance question and a local truth question to present to each user accessing the ingested recommendation;
generating metadata for storage within one or more web servers for use in generating and rendering to a user the ingested recommendation upon an electronic device associated with the user.

17. The method according to claim 16, further comprising:
providing a user with credits, each credit associated with the user accessing and engaging within a software application with an ingested recommendation; wherein
the credits are fed back from the software application to at least one of a professional association associated with the user and an electronic medical record of the user;
each credit is established in dependence upon a response of the user with respect to at least one of the local relevance question and the local truth question associated with the recommendation;
when the credits are fed back to the professional association the credits are employed either in establishing an accreditation of the user with the professional association or in completing a requirement for advancement of the user with the professional association; and
when the credits are fed back to the electronic medical record of the user the credits are subsequently employed by a medical professional to establish a history that defines the user's background knowledge or experience with “plain language” recommendations.

18. (canceled)

19. The method according to claim 16, further comprising:
providing a user with credits, each credit associated with the user accessing and engaging within a software application a recommendation; wherein
the credits are linked from the software application to another software application; and
the user can establish at least one of:
at least one of a financial reward, a financial discount, and a financial credit to employ in respect of purchasing at least one of a service and a product; and
an option with respect of at least one of a service and a product which is only available to users exceeding a specific credit threshold.

20. A system comprising:
a memory accessible to one or more microprocessors storing a database comprising a plurality of recommendations;
another memory accessible to the one or more microprocessors storing another database comprising user data relating to a set of users;
a further memory accessible to the one or more microprocessors storing computer executable instructions which when executed by the one or more microprocessors configure the one or more microprocessors to execute a process comprising:
managing documentation for each cluster of a plurality of clusters;
managing communications between users for each cluster of the plurality of users; and
establish one or more knowledge-based communities for each cluster of the plurality of clusters; wherein
each cluster of the plurality of clusters is established in dependence upon at least one of:
geographic locations of each user of a plurality of users accessing the system; and
profiles of each user of the plurality of users accessing the systems.

21. The system according to claim 20, wherein
at least one of:
each cluster of the plurality of clusters has different at least one of inferences, influences and knowledge; and
each knowledge-based communities for each cluster of the plurality of clusters is established in dependence upon the profiles of each user within a subset of the plurality of users where the subset of the plurality of users are those users within the cluster of the plurality of clusters.

22. (canceled)

23. The method according to claim 16, wherein
the credits are fed back from the software application to at least one of a professional association associated with the user and an electronic medical record of the user;
the credit is modified by a scaling; and
the scaling is established in dependence upon one or more factors selected from the group comprising:
does the user answer the at least one of the local relevance question and the local truth question associated with the recommendation a question correctly or not;
a degree of accuracy of the user's answer to the at least one of the local relevance question and the local truth question associated with the recommendation;
a degree of accuracy of the user's answer to the at least one of the local relevance question and the local truth question associated with the recommendation where an answer comprises multiple elements;
how many of at least one of the local relevance question and the local truth question associated with a plurality of recommendations the user answers;
how quickly the user answers the at least one of the local relevance question and the local truth question associated with the recommendation;
a subsequent repetition of the at least one of the local relevance question and the local truth question associated with the recommendation and adjusting the scaling based upon a degree of improvement or degradation in the accuracy of the responses from the user; and
does the user skip the at least one of the local relevance question and the local truth question associated with the recommendation by default.
